(19) United States
(12) Patent Application Publication
(10) Pub. No.: US 2011/0045080 A1
(43) Pub. Date: Feb. 24, 2011

(54) Single-Walled Carbon Nanotube/Bioactive Substance Complexes and Methods Related Thereto

(75) Inventors: Garth Powis, Houston, TX (US); Jeffrey Bartholomeusz, Houston, TX (US); James Tour, Bellaire, TX (US); Howard Schmidt, Cypress, TX (US); Paul Cherukuri, Houston, TX (US); R. Bruce Weisman, Houston, TX (US)

Correspondence Address: WINSTEAD PC, P.O. BOX 50784, DALLAS, TX, 75201, US

(73) Assignee: WILLIAM MARSH RICE UNIVERSITY, Houston, TX (US)

(21) Appl. No.: 12/731,021

(22) Filed: Mar. 24, 2010

Related U.S. Application Data
(60) Provisional application No. 61/162,933, filed on Mar. 24, 2009.

(51) Int. Cl. A61K 9/14; A61K 31/713; C07H 21/02; A61P 35/00; A61P 29/00; C12N 5/00; B82Y 5/00

(52) U.S. Cl. 424/489; 514/44 A; 536/24.5; 435/375; 977/750

(57) ABSTRACT

The present invention includes single-walled carbon nanotube compositions for the delivery of siRNA and methods of making such single-walled carbon nanotube compositions. A single-walled carbon nanotube composition for delivery of siRNA includes a nonfunctionalized single-walled carbon nanotube; and siRNA noncovalently complexed with the nonfunctionalized single-walled carbon nanotube, wherein the siRNA solubilizes such nonfunctionalized single-walled carbon nanotube.

### CROSS-REFERENCE TO RELATED APPLICATIONS

The present application claims priority to provisional application No. 61/162,933 filed on Mar. 24, 2009 which is incorporated by reference herein in its entirety.

### STATEMENT REGARDING FEDERALLY SPONSORED RESEARCH

This invention was made during work supported by the NIH (CA-52995-18, CA-77204, CA-98920 and CA-109552), the NSF Center for Biological and Environmental Nanotechnology (EEC-0647452) and the Alliance for NanoHealth (NASA JSC-NNJ06HC25G). The Government has certain rights in the invention.

### BACKGROUND OF THE INVENTION

The invention presented herein relates to gene therapy systems. More specifically, the present invention relates to nonfunctionalized single-walled carbon nanotubes coated with bioactive agents and methods related thereto.

Gene therapy has become an increasingly important mode of treatment for a variety of indications. RNA interference (RNAi), in particular, is a promising treatment method. RNA interference (RNAi) or gene silencing involves reducing the expression of a target gene through mediation by small single- or double-stranded RNA molecules. These molecules include small interfering RNAs (siRNAs), microRNAs (miRNAs), and small hairpin RNAs (shRNAs), among others.

Numerous gene therapy platforms for the delivery of such molecules are currently available. Within the family of nanotechnology-based gene therapy platforms are carbon nanotubes (CNTs). CNTs can be functionalized to deliver their cargos to cells and organs. However, typically before CNTs can be used in biomedical applications, the hydrophobic nonfunctionalized CNTs must be suspended in aqueous solutions.

### SUMMARY OF THE INVENTION

Embodiments of the present invention provide a single-walled carbon nanotube (SWCNT) composition for delivery of a bioactive agent, including a nonfunctionalized SWCNT and a bioactive substance noncovalently complexed with the nonfunctionalized SWCNT, wherein the bioactive substance solubilizes such nonfunctionalized SWCNT. In certain embodiments, the nonfunctionalized SWCNT is unagglomerated and nonaggregated. The terms “unagglomerated” and “nonaggregated” are defined in the specification below.

The SWCNTs of embodiments of the present invention may be of any diameter, such as, for example, about 0.01 nm to about 2 nm, about 0.05 nm to about 1.5 nm, and about 0.1 nm to about 1 nm. In another embodiment, the diameter may be about 1 nm. In yet another embodiment, the diameter may be about 1 nm to about 2 nm.

The length of the SWCNTs of embodiments of the present invention may be any length, but in particular embodiments, the length is about 1 nm to about 500 nm, about 5 nm to about 450 nm, about 10 nm to about 400 nm, about 50 nm to about 350 nm, about 100 nm to about 300 nm, and about 150 nm to about 250 nm. In other embodiments, the length is about 125 nm to about 275 nm, and about 175 nm to about 225 nm. In some embodiments, the length of the SWCNT may be about 500 nm or less. In other embodiments, the length is less than about 400 nm. In preferred embodiments, the length is about 100 nm to about 300 nm.

As used herein, the term “bioactive substance” means a compound utilized to image, impact, treat, combat, ameliorate, prevent or improve an unwanted condition or disease of a patient. The bioactive substance may be any bioactive substance known to those of ordinary skill in the art. In preferred embodiments, the bioactive substance is siRNA.

Non-limiting examples of bioactive substances include chemotherapeutic agents, diagnostic agents, prophylactic agents, nutraceutical agents, nucleic acids, proteins, peptides, lipids, carbohydrates, hormones, small molecules, metals, ceramics, vaccines, immunological agents, and combinations thereof. In some embodiments, the bioactive substance is a “drug.” A “drug” is defined herein to refer to any substance that is known or suspected to be of benefit in the treatment, prevention, or diagnosis of a disease or health-related condition.

Non-limiting examples of diseases or health-related conditions include immune diseases, inflammatory diseases, degenerative diseases, hyperproliferative diseases, infectious diseases, trauma, malnutrition, and so forth. An example of a hyperproliferative disease is cancer. Non-limiting examples of cancer include skin cancer, cancer of the head and neck, stomach cancer, intestinal cancer, pancreatic cancer, liver cancer, colon cancer, prostate cancer, ovarian cancer, uterine cancer, renal cancer, lung cancer, leukemia, and breast cancer. In one or more preferred embodiments, the bioactive substance includes siRNA. In some aspects of the invention, the bioactive substance includes chemically-modified siRNA. In certain aspects of the invention, the bioactive substance includes “non-targeting siRNA,” meaning siRNA used for non-sequence-specific effects. In other aspects, the bioactive substance includes “targeting siRNA” wherein the siRNA is targeted to mRNA.

The targeting siRNA may be targeted to any mRNA. In a non-limiting example, the siRNA is targeted to hypoxia-inducible factor 1 alpha (HIF-1α) mRNA. In other embodiments, the siRNA is targeted to vascular endothelial growth factor (VEGF) mRNA, in which case the sense strand of the siRNA may be AUGUGAAUGCAGACCAAAGAA (SEQ ID NO:1), among others. The siRNA of other embodiments is targeted to endothelial growth factor receptor (EGFR) mRNA, in which case the sense strand may be GUCAGCCUGAACAUAACAU (SEQ ID NO:2) or GUGUAACGGAAUAGGUAUU (SEQ ID NO:3), among others. The siRNA of yet other embodiments is targeted to human epidermal growth factor receptor 2 (HER2) mRNA. In this case, the sense strand of the siRNA may be GGAGCUGGCGGCCUUGUGCCG (SEQ ID NO:4) or UCACAGGGGCCUCCCCAGGAG (SEQ ID NO:5), among others.

In certain aspects of the present invention, the SWCNT complexes may be optimized with a specific ratio of complexed to noncomplexed surface area, such that the SWCNTs are solubilized into solution and a therapeutically effective amount of bioactive agent is delivered. Any amount of surface area of the SWCNT may be complexed with the bioactive substance or mixture of bioactive substances. For example, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99%, or about 100% of the surface area of the SWCNT may be complexed with one or more bioactive substances, or any range of surface areas derivable therein may be complexed with one or more bioactive substances.

Some embodiments hereof provide a SWCNT composition including a nonfunctionalized SWCNT and a bioactive substance noncovalently solubilizing such nonfunctionalized SWCNT. The SWCNT composition may be internalized in treated cells in media containing 10% serum at a rate measured in vitro that substantially corresponds to the following: (i) from about 0.01% to about 30% of the total amount of treated cells internalize the single-walled carbon nanotube composition after about 1 hour of measurement; (ii) from about 20% to about 90% of the total amount of treated cells internalize the single-walled carbon nanotube composition after about 3 hours of measurement; and (iii) not less than about 95% of the total amount of treated cells internalize the single-walled carbon nanotube composition after about 24 hours of measurement. In some embodiments, the bioactive agent dissociates from the SWCNT when internalized in the treated cell. In other embodiments, the bioactive agent remains complexed with the SWCNT when internalized in the treated cell.

Other embodiments hereof provide a SWCNT composition including a nonfunctionalized SWCNT and a bioactive substance noncovalently solubilizing such nonfunctionalized SWCNT wherein the SWCNT composition is internalized in a treated cell in media containing 10% serum at a rate measured in vitro that substantially corresponds to the following: (i) from about 0.01% to about 30% of the total SWCNT composition is internalized after about 1 hour of measurement; (ii) from about 20% to about 90% of the total SWCNT composition is internalized after about 3 hours of measurement; and (iii) not less than about 95% of the total SWCNT composition is internalized after about 24 hours of measurement. In some embodiments, the bioactive agent dissociates from the SWCNT when internalized in the treated cell. In other embodiments, the bioactive agent remains complexed with the SWCNT when internalized in the treated cell.

Some aspects of the present invention include a pharmaceutical composition that includes a nonfunctionalized SWCNT, a bioactive agent noncovalently complexed with the nonfunctionalized SWCNT, and a pharmaceutically acceptable carrier. In preferred embodiments of the present invention, the bioactive agent is an siRNA. The nonfunctionalized SWCNT is solubilized into the pharmaceutically acceptable carrier by association with the siRNA. In preferred embodiments, the pharmaceutically acceptable carrier is liquid. The pharmaceutically acceptable carrier may be any liquid. Non-limiting examples include water and an isotonic solution, such as an isotonic salt solution or an isotonic sugar solution. The pharmaceutically acceptable carrier of further aspects is aqueous polyethylene glycol (PEG) solution. In yet others, the carrier includes an organic solvent dissolved in isotonic aqueous solution. In yet other aspects, the pharmaceutically acceptable carrier is an aqueous buffer solution.

The final concentration of nonfunctionalized SWCNT may be any concentration, such as about 1 μg/L, about 100 μg/L, about 200 μg/L, about 300 μg/L, about 400 μg/L, about 500 μg/L, about 600 μg/L, about 700 μg/L, about 800 μg/L, about 900 μg/L, about 1 mg/L, about 1.2 mg/L, about 1.4 mg/L, about 1.6 mg/L, about 1.8 mg/L, about 2.0 mg/L, about 2.2 mg/L, about 2.4 mg/L, about 2.6 mg/L, about 2.8 mg/L, about 3.0 mg/L, about 3.2 mg/mL, about 3.4 mg/L, about 3.6 mg/L, about 3.8 mg/L, about 4.0 mg/L, about 4.2 mg/L, about 4.4 mg/L, about 4.6 mg/L, about 4.8 mg/L, about 5.0 mg/L, about 5.2 mg/L, about 5.4 mg/L, about 5.6 mg/L, about 5.8 mg/L, about 6.0 mg/L, about 6.5 mg/L, about 7.0 mg/L, about 7.5 mg/L, about 8.0 mg/L, about 8.5 mg/L, about 9.0 mg/L, about 9.5 mg/L, about 10.0 mg/L, about 15 mg/L, about 20 mg/L, about 25 mg/L, about 30 mg/L, about 35 mg/L, about 40 mg/L, about 45 mg/L, about 50 mg/L, about 60 mg/L, about 70 mg/L, about 80 mg/L, about 90 mg/L, about 100 mg/L, about 200 mg/L, about 300 mg/L, about 400 mg/L, about 500 mg/L or greater, or any range of concentrations of nonfunctionalized SWCNT derivable herein.

The final concentration of bioactive agent in the composition may be any concentration, such as about 0.001 μM, about 0.005 μM, about 0.010 μM, about 0.02 μM, about 0.03 μM, about 0.04 μM, about 0.05 μM, about 0.06 μM, about 0.07 μM, about 0.08 μM, about 0.09 μM, about 0.1 μM, about 0.2 μM, about 0.3 μM, about 0.4 μM, about 0.5 μM, about 0.6 μM, about 0.7 μM, about 0.8 μM, about 0.9 μM, about 1.0 μM, about 1.1 μM, about 1.25 μM, about 1.5 μM, about 1.75 μM, about 2.0 μM, about 2.25 μM, about 2.5 μM, about 2.75 μM, about 3.0 μM, about 3.25 μM, about 3.5 μM, about 3.75 μM, about 4.0 μM, about 4.25 μM, about 4.5 μM, about 4.75 μM, about 5.0 μM, about 5.5 μM, about 6.0 μM, about 6.5 μM, about 7.0 μM, about 7.5 μM, about 8.0 μM, about 8.5 μM, about 9.0 μM, about 9.5 μM, about 10 μM, about 12 μM, about 15 μM, about 20 μM, about 30 μM, about 35 μM, about 40 μM, about 50 μM, about 60 μM, about 70 μM, about 80 μM, about 85 μM, about 90 μM, about 100 μM, about 200 μM, about 300 μM, about 400 μM, about 500 μM, about 1 mM, about 1.5 mM, about 2.0 mM, about 2.5 mM, about 3.0 mM, about 5 mM, about 10 mM, about 25 mM, about 50 mM, about 75 mM, about 100 mM, about 500 mM, about 100 mM or greater, or any range of concentrations of bioactive agent derivable therein. In some aspects of the present invention, the final concentrations of the pharmaceutical composition are 3 mg/L nonfunctionalized SWCNT and about 5 μM siRNA.

In one or more embodiments, the pharmaceutical composition provides delivery of an effective amount of siRNA. In certain embodiments, the “effective amount” is that amount that reduces the expression of a target nucleic acid when compared to a strand of siRNA not complexed to the nonfunctionalized SWCNT.

Embodiments hereof provide a method of reducing the expression of a targeted gene in cell culture, including delivering an effective amount of a SWCNT composition comprising a nonfunctionalized single-walled carbon nanotube and a bioactive substance noncovalently complexed with the nonfunctionalized SWCNT wherein the bioactive substance solubilizes such nonfunctionalized SWCNT.

In other embodiments, a method of effectively silencing a targeted gene in vivo is provided, including administering to a subject an effective amount of a SWCNT composition comprising a nonfunctionalized SWCNT and a bioactive substance noncovalently complexed with the nonfunctionalized SWCNT wherein the bioactive substance solubilizes such nonfunctionalized SWCNT.

In yet further embodiments, a method for preparing a SWCNT composition is provided, including providing a dry nonfunctionalized SWCNT, providing a siRNA solution, adding the dry nonfunctionalized SWCNT to the siRNA solution and sonicating the nonfunctionalized SWCNT in the siRNA solution. The step of providing the siRNA solution may comprise resuspending siRNA in solution.

In still other embodiments, a method for preparing a single-walled carbon nanotube composition is provided including providing a dry nonfunctionalized single-walled carbon nanotube, providing a solution comprising one or more bioactive agents, adding the solution to the dry nonfunctionalized single-walled carbon nanotube, and sonicating the nonfunctionalized single-walled carbon nanotube in the solution. The bioactive agent may be any bioactive agent as set forth in this disclosure. In preferred embodiments, the bioactive agent is a siRNA.

It is specifically contemplated that any limitation discussed with respect to one embodiment of the invention may apply to any other embodiment of the invention. Furthermore, any composition of the invention may be used in any method of the invention, and any method of the invention may be used to produce or to utilize any composition of the invention.

The use of the term “or” in the claims is used to mean “and/or” unless explicitly indicated to refer to alternatives only or the alternative are mutually exclusive, although the disclosure supports a definition that refers to only alternatives and “and/or.”

As used herein the specification, “a” or “an” may mean one or more, unless clearly indicated otherwise. As used herein in the claim(s), when used in conjunction with the word “comprising,” the words “a” or “an” may mean one or more than one. As used herein “another” may mean at least a second or more.

As used herein, the term “about” means plus or minus 10% of the numerical value of the number with which it is being used. Therefore, about 50% means in the range of 45%-55%.

The terms “include,” “comprise” and “have” and their conjugates, as used herein, mean “including but not necessarily limited to.”

Other objects, features and advantages of the present invention will become apparent from the following detailed description. It should be understood, however, that the detailed description and the specific examples, while indicating preferred embodiments of the invention, are given by way of illustration only, as various changes and modifications within the spirit and scope of the invention will become apparent to those skilled in the art from this detailed description.

Additional features and advantages of the invention will become apparent from the following drawings and detailed description.

### BRIEF DESCRIPTION OF THE FIGURES

The following figures form part of the present specification and are included to further demonstrate certain aspects of the present invention. The invention may be better understood by reference to one or more of these figures in combination with the detailed description of specific embodiments presented herein.

FIG. 1A depicts nonfunctionalized single-walled carbon nanotubes (SWCNTs) in solution;

FIG. 1B illustrates siRNA-solubilized SWCNT solution;

FIG. 1C is a normalized emission spectra (using 658 nm excitation) of nonfunctionalized SWCNTs solubilized with siRNA;

FIG. 2 includes bright field and near-IR (NIR) images of incubated cells with internalized SWCNTs;

FIG. 3 graphically depicts the cell viability of MiaPaCa-HRE pancreatic cancer cells after delivery of biologically active siRNA via SWCNTs;

FIGS. 4A and 4B graphically depict inducement of RNA interference (RNAi) response after delivery of siRNA into cells by nonfunctionalized SWCNTs;

FIG. 4A graphically depicts the inhibition of HIF-Iα activity in cells treated with the SWCNT-siHIF-1α complex as determined by luciferase assay;

FIG. 4B graphically depicts the inhibition of HIF-Iα protein expression by Western blotting;

FIG. 5 graphically illustrates siRNA delivered into a variety of cancer cells by nonfunctionalized SWCNTs induces RNAi response with similar efficiency;

FIGS. 6A-6E illustrate the inhibition of HIF-Iα activity in a xenograft mouse tumor after administration of SWCNT/siRNA complexes;

FIG. 6A graphically depicts the cell viability of MiaPaCa-HRE pancreatic cancer cells after delivery of a range of concentrations of SWCNT/siRNA complexes;

FIGS. 6B and 6C are images of tumor bearing mice given intratumoral injections of either siRNA targeting HIF-α alone (siHIF-Iα), a non-targeting siRNA complexed to SWCNTs (SWCNT/siSc), or siRNA targeting HIF-1α complexed to SWCNTs (SWCNT-siHIF) twice per week for 3 weeks;

FIG. 6D graphically depicts decreased tumor HIF-Iα activity in mice treated with SWCNT/HIF complexes compared to mice treated with complexes comprising either the control SWCNT/siRNA (p<0.01 to p<0.05) or HIF-1α siRNA alone; and

FIG. 6E graphically depicts tumor volume as a function of days after cell injection of SWCNT/siRNA complexes.

### DESCRIPTION OF ILLUSTRATIVE EMBODIMENTS

The present invention is in part based on the finding that a single-walled carbon nanotube (SWCNT) composition can be applied in the delivery of a bioactive agent. In some aspects, for example, SWCNT may be a nonfunctionalized SWCNT that includes one or more bioactive substances noncovalently complexed with the nonfunctionalized SWCNT, wherein the bioactive substance solubilizes such nonfunctionalized SWCNT. This invention is not limited to the particular compositions or methodologies described, as these may vary. In addition, the terminology used in the description describes particular versions or embodiments only and is not intended to limit the scope of the present invention. Unless defined otherwise, all technical and scientific terms used herein have the same meanings as commonly understood by one of ordinary skill in the art. In case of conflict, the patent specification, including definitions, will prevail.

### A. Carbon Nanotube and Carbon Nanotube Compositions

Some embodiments of the present invention provide a single-walled carbon nanotube (SWCNT) composition for delivery of a bioactive agent including a nonfunctionalized SWCNT and a bioactive substance noncovalently complexed with the nonfunctionalized SWCNT, wherein the bioactive substance solubilizes such nonfunctionalized SWCNT. In some embodiments, the bioactive substance also disperses the SWCNT.

### 1. Definitions

The term “carbon nanotube,” as used herein, refers to a tube that contains a sheet of graphene rolled into a cylinder. The term carbon nanotube refers to both single-walled nanotubes (SWNTs) and multiwalled nanotubes (MWNTs), with many concentric shells. The term carbon nanotube, as used herein, may further include structures that are not entirely carbon, such as metals, small-gap semiconductors or large-gap semiconductors. For example, boron carbon nitride (BCN) nanotubes are included in the definition of carbon nanotube. The present carbon nanotubes may also be graphene in other forms. This includes, for example, a single sheet of graphene formed into a sphere, which constitutes a carbon nanosphere, commonly referred to as a buckyball or fullerene. The carbon nanotubes may be produced by any method known to those of ordinary skill in the art. Non-limiting examples of methods for the production of carbon nanotubes include arc discharge, laser ablation and chemical vapor deposition.

The term “nonfunctionalized,” as used herein, refers to pristine SWCNTs. In some embodiments, pristine SWCNTs include SWCNTs with surfaces that are unmodified in that the SWCNT surfaces have not been associated with a functional group such as, for example, a linking group that links the SWCNT surfaces with siRNA.

The terms “stable” and “stabilized,” as used herein, mean a solution or suspension in a fluid phase wherein solid components (i.e., nanotubes and bioactive substances) possess stability against aggregation and agglomeration sufficient to allow manufacture and delivery to a cell and which maintain the integrity of the compound for a sufficient period of time to be detected and preferably for a sufficient period of time to be useful for the purposes detailed herein.

The terms “agglomerated” and “agglomeration,” as used herein, refer to the formation of a cohesive mass consisting of carbon nanotubes held together by relatively weak forces (for example, van der Waals or capillary forces) that may break apart into subunits upon processing, for example. The resulting structure is called an “agglomerate.” The term “unagglomerated,” as used herein, means the opposite of “agglomerated” and refers to a state of dispersion of carbon nanotubes in that the carbon nanotubes are not held together.

As used herein, the terms “aggregated” and “aggregation” refer to the formation of a discrete group of carbon nanotubes in which the various individual carbon nanotubes are not easily broken apart, such as in the case of nanotube bundles that are strongly bonded together. The resulting structure is called an “aggregate.” The terms “nonaggregated” or “unaggregated,” as used herein, mean the opposite of “aggregated” and refers to a state of dispersion of carbon nanotubes in that the carbon nanotubes are not held together.

### 2. Methods of Preparation of SWCNT Compositions

In some embodiments of the present invention, a method for preparing a SWCNT composition is provided including providing a dry nonfunctionalized SWCNT, providing a siRNA solution, adding the dry nonfunctionalized SWCNT to the siRNA solution and sonicating the nonfunctionalized SWCNT in the siRNA solution. Formation of the SWCNT/siRNA noncovalent complexes requires only ultrasonic agitation, rather than chemical reaction. The step of providing the siRNA solution may comprise resuspending siRNA in solution. In other embodiments, a method for preparing a single-walled carbon nanotube composition is provided including providing a dry nonfunctionalized single-walled carbon nanotube, providing a siRNA solution, adding the siRNA solution to the dry nonfunctionalized single-walled carbon nanotube, and sonicating the nonfunctionalized single-walled carbon nanotube in the siRNA solution.

### B. Inhibition of Gene Expression

Preferred embodiments of the present invention are SWCNT compositions and methods related thereto that include siRNA as the bioactive substance. In these embodiments, formation of the SWCNT/siRNA noncovalent complexes requires only ultrasonic agitation, rather than chemical reaction. In addition, the siRNA in these complexes retain biological activity and readily enter cells, even in the presence of serum.

### 1. Definitions

“Gene silencing” refers to the suppression of gene expression, e.g., transgene, heterologous gene and/or endogenous gene expression. Gene silencing may be mediated through processes that affect transcription and/or through processes that affect post-transcriptional mechanisms. In some embodiments, gene silencing occurs when siRNA initiates the degradation of the mRNA of a gene of interest in a sequence-specific manner via RNA interference. Certain embodiments hereof provide a method of reducing the expression of a targeted gene in cell culture, including delivering an effective amount of a SWCNT composition comprising a nonfunctionalized single-walled carbon nanotube and a bioactive substance noncovalently complexed with the nonfunctionalized SWCNT wherein the bioactive substance solubilizes such nonfunctionalized SWCNT.

“Knock-down” or “knock-down technology” refers to a technique of gene silencing in which the expression of a target gene is reduced as compared to the gene expression prior to the introduction of the siRNA, which can lead to the inhibition of production of the target gene product.

“RNA interference (RNAi)” is the process of sequence-specific, posttranscriptional gene silencing initiated by siRNA. RNAi is seen in a number of organisms such as Drosophila, nematodes, fungi and plants, and is believed to be involved in anti-viral defense, modulation of transposon activity, and regulation of gene expression. During RNAi, siRNA induces degradation of target mRNA with consequent sequence-specific inhibition of gene expression.

The terms “small interfering” or “short interfering RNA” or “siRNA” refer to a RNA duplex of nucleotides that is targeted to a gene of interest. A “RNA duplex” refers to the structure formed by the complementary pairing between two regions of a RNA molecule. siRNA is “targeted” to a gene in that the nucleotide sequence of the duplex portion of the siRNA is complementary to a nucleotide sequence of the targeted gene. In some embodiments, the length of the duplex of siRNA is less than 30 nucleotides. In some embodiments, the duplex can be 29, 28, 27, 26, 25, 24, 23, 22, 21, 20, 19, 18, 17, 16, 15, 14, 13, 12, 11 or 10 nucleotides in length. In some embodiments, the length of the duplex is 19-25 nucleotides in length. The RNA duplex portion of the siRNA can be part of a hairpin structure. In addition to the duplex portion, the hairpin structure may contain a loop portion positioned between the two sequences that form the duplex. The loop can vary in length. In some embodiments the loop is 5, 6, 7, 8, 9, 10, 11, 12 or 13 nucleotidesweeks in length. The hairpin structure can also contain 3′ or 5′ overhang portions. In some embodiments, the overhang is a 3′ or a 5′ overhang 0, 1, 2, 3, or 5 nucleotides in length. In some embodiments, siRNA refers to a class of doublestranded RNA molecules including, for example, chemically-modified siRNA, stabilized siRNA, targeting siRNA, and non-targeting siRNA.

siRNA can be obtained from commercial sources, natural sources, or can be synthesized using any of a number of techniques well-known to those of ordinary skill in the art.

Preferably, RNAi is capable of decreasing the expression of a particular protein, by at least 10%, 20%, 30%, or 40%, more preferably by at least 50%, 60%, or 70%, and most preferably by at least 75%, 80%, 90%, 95% or more.

### C. Treatment and Prevention of Disease

One aspect of the invention includes methods for treating or preventing a disease using single-wall carbon nanotube compositions as set forth herein. The diseases that may be treated using methods of the present invention encompass a broad range of indications. For example, as SWCNT complexes of embodiments of the present invention have the potential to function as a serum-insensitive, wide range transfection agent to deliver bioactive agents such as siRNA into cells to induce a response. The SWCNT complexes can be used for a variety of applications, such as, without limitation, drug delivery, gene therapy, medical diagnosis and for medical therapeutics for cancer, pathogen-borne diseases, hormone-related diseases, reaction-by-products associated with organ transplants, and other abnormal cell or tissue growth.

### 1. Definitions

“Treatment” and “treating” refer to administration or application of SWCNT complexes to a subject or performance of a procedure or modality on a subject for the purpose of obtaining a therapeutic benefit of a disease or health-related condition.

A “subject” refers to either a human or non-human, such as primates, mammals, and vertebrates. In particular embodiments, the subject is a human. The term “patient,” as used herein, includes human and veterinary subjects.

The term “diseased tissue,” as used herein, refers to tissue or cells associated with solid tumor cancers of any type, such as bone, lung, vascular, neuronal, colon, ovarian, breast and prostate cancer. The term diseased tissue may also refer to tissue or cells of the immune system, such as tissue or cells effected by AIDS; pathogen-borne diseases, which can be bacterial, viral, parasitic, or fungal, examples of pathogen-borne diseases include HIV, tuberculosis and malaria; hormone-related diseases, such as obesity; vascular system diseases; central nervous system diseases, such as multiple sclerosis; and undesirable matter, such as adverse angiogenesis, restenosis amyloidosis, toxins, reaction-by-products associated with organ transplants, and other abnormal cell or tissue growth.

An “effective amount” or “therapeutically effective amount” of a composition, as used herein, refers to an amount of a biologically active molecule or complex or derivative thereof sufficient to exhibit a detectable therapeutic effect without undue adverse side effects (such as toxicity, irritation and allergic response) commensurate with a reasonable benefit/risk ratio when used in the manner of the invention. The therapeutic effect may include, for example but not by way of limitation, inhibiting the growth of undesired tissue or malignant cells. The effective amount for a subject will depend upon the type of subject, the subject's size and health, the nature and severity of the condition to be treated, the method of administration, the duration of treatment, the nature of concurrent therapy (if any), the specific formulations employed, and the like.

The term “therapeutic benefit” or “therapeutically effective” as used throughout this application refers to anything that promotes or enhances the well-being of the subject with respect to the medical treatment of this condition. This includes, but is not limited to, a reduction in the frequency or severity of the signs or symptoms of a disease. For example, treatment of cancer may involve, for example, a reduction in the size of a tumor, a reduction in the invasiveness of a tumor, reduction in the growth rate of the cancer, or prevention of metastasis. Treatment of cancer may also refer to prolonging survival of a subject with cancer.

In some embodiments of the invention, the methods include identifying a patient in need of treatment. A patient may be identified, for example, based on taking a patient history, based on findings on clinical examination, based on health screenings, or by self-referral.

### 2. Bioactive Substances

The bioactive substance may be any such substance known to those of ordinary skill in the art. In certain embodiments it is selected from the group consisting of chemotherapeutic agents, diagnostic agents, prophylactic agents, nutraceutical agents, nucleic acids, proteins, peptides, lipids, carbohydrates, hormones, small molecules, metals, ceramics, drugs, vaccines, immunological agents, and combinations thereof. In one or more preferred embodiments, the bioactive substance comprises siRNA. Numerous siRNA sequences can be utilized to complex the nonfunctionalized SWCNTs. Further, in some aspects of the invention, siRNA solubilizes the SWCNTs equally effectively, irrespective of nucleotide sequences. In certain aspects of the invention, the bioactive substance comprises chemically-modified siRNA. In other aspects, the bioactive substance comprises non-targeting siRNA. In yet other aspects, the bioactive substance comprises targeting siRNA. The siRNA in certain embodiments is targeted to hypoxia-inducible factor 1 alpha (HIF-1α).

### 3. Diseases

A “disease” or “health-related condition” can be any pathological condition of a body part, an organ, or a system resulting from any cause, such as infection, genetic defect, and/or environmental stress. The cause may or may not be known. The present invention may be used to treat or prevent any disease or health-related condition in a subject. Examples of such diseases have been previously set forth, and include infectious diseases, inflammatory diseases, hyperproliferative diseases such as cancer, degenerative diseases, and so forth. For example, SWCNT complexes of the invention may be administered to treat a cancer. The cancer may be a solid tumor, metastatic cancer, or non-metastatic cancer. In certain embodiments, the cancer may originate in the bladder, blood, bone, bone marrow, brain, breast, colon, esophagus, duodenum, small intestine, large intestine, colon, rectum, anus, gum, head, kidney, liver, lung, nasopharynx, neck, ovary, prostate, skin, stomach, testis, tongue, or uterus. In certain embodiments, the cancer is colorectal cancer (i.e., cancer involving the colon or rectum).

The cancer may specifically be of the following histological type, though it is not limited to these: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; cerummous adenocarcinoma; mucoepidermoid carcinoma; cystadenocarcinoma; papillary cystadenocarcinoma; papillary serous cystadenocarcinoma; mucinous cystadeno carcinoma; mucinous adenocarcinoma; signet ring cell carcinoma; infiltrating duct carcinoma; medullary carcinoma; lobular carcinoma; inflammatory carcinoma; paget's disease, mammary; acmar cell carcinoma; adenosquamous carcinoma; adenocarcinoma w/squamous metaplasia; thymoma, malignant; ovarian stromal tumor, malignant; thecoma, malignant; granulosa cell tumor, malignant; androblastoma, malignant; sertoli cell carcinoma; leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; malignant melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangiosarcoma; hemangioendothelioma, malignant; kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; hodgkin's disease; hodgkin's; paragranuloma; malignant lymphoma, small lymphocytic; malignant lymphoma, large cell, diffuse; malignant lymphoma, follicular; mycosis fungoides; other specified non-hodgkin's lymphomas; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; and hairy cell leukemia. Nonetheless, it is also recognized that the present invention may also be used to treat a non-cancerous disease (e.g., a fungal infection, a bacterial infection, a viral infection, and/or a neurodegenerative disease). In a specific embodiment, the cancer is pancreatic cancer.

### D. Pharmaceutical Preparations

In some embodiments, a method of treating or preventing disease in a subject or imaging a subject is provided, including administering to a subject an effective amount of a SWCNT composition comprising a nonfunctionalized SWCNT and a bioactive substance noncovalently complexed with the nonfunctionalized SWCNT wherein the bioactive substance solubilizes such nonfunctionalized SWCNT. In preferred embodiments, the bioactive substance is a siRNA. The results demonstrate that siRNA can be used to solubilize nonfunctionalized SWCNTs and that noncovalent SWCNT/siRNA complexes can transfect cancer cells and effectively silence a targeted gene in cell culture and also in tumors in vivo. In other aspects of the present invention, siRNA can be used to silence target genes with a high degree of specificity. For example, intra-tumoral administration of SWCNT/siRNA complexes targeting HIF-1α significantly reduces HIF-1α activity in tumor-bearing mice.

Where clinical application of the SWCNT complexes of the present invention is undertaken, it will generally be beneficial to prepare the SWCNT complexes as a pharmaceutical composition appropriate for the intended application. This will typically entail preparing a pharmaceutical composition that is essentially free of pyrogens, as well as any other impurities that could be harmful to humans or animals. In preparing a pharmaceutical composition, one may also employ appropriate buffers to render the complex stable and allow for uptake by target cells.

The phrases “pharmaceutically acceptable” and “pharmacologically acceptable” refer to molecular entities and compositions that do not produce an adverse, allergic or other untoward reaction when administered to an animal, such as a human, as appropriate. The preparation of a pharmaceutical composition that contains at least one non-charged lipid component comprising a siRNA or additional active ingredient is exemplified by Remington: The Science and Practice of Pharmacy, 21^{st} Edition, 2005, which is incorporated herein by reference. Moreover, for animal and human administration, it will be understood that preparations should meet sterility, pyrogenicity, general safety and purity standards as required by FDA Office of Biological Standards.

As used herein, “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drugs, drug stabilizers, gels, binders, excipients, disintegration agents, lubricants, sweetening agents, flavoring agents, dyes, such like materials and combinations thereof, as would be known to one of ordinary skill in the art. A pharmaceutically acceptable carrier is preferably formulated for administration to a human, although in certain embodiments it may be desirable to use a pharmaceutically acceptable carrier that is formulated for administration to a non-human animal but which would not be acceptable (e.g., due to governmental regulations) for administration to a human. Except insofar as any conventional carrier is incompatible with the active ingredient, its use in the therapeutic or pharmaceutical compositions is contemplated.

In preferred embodiments, the pharmaceutically acceptable carrier is liquid. Examples of pharmaceutically acceptable carriers that may be utilized in accordance with the present invention include, but are not limited to, water, isotonic salt solution, isotonic sugar solution, polyethylene glycol (PEG), aqueous PEG solutions, propylene glycol, injectable organic esters such as ethyloleate, liposomes, ethanol, organic solvent (e.g. DMSO) dissolved in isotonic aqueous solution, alcoholic/aqueous solutions, parenteral vehicles such as sodium chloride, Ringer's dextrose, aqueous buffers, oils, and combinations thereof. Under ordinary conditions of storage and use, these preparations may contain a preservative to prevent the growth of microorganisms, etc. Non-limiting examples of preservatives include antimicrobial agents, anti-oxidants, chelating agents and inert gases. The pH and exact concentration of the various components the pharmaceutical composition are adjusted according to well known parameters.

As would be appreciated by one of skill in this art, the carrier may be selected based on factors including, but not limited to, route of administration, location of the disease tissue, the bioactive substance being delivered, and/or time course of delivery of the bioactive substance. The pharmaceutically acceptable carrier solution in certain embodiments is water. In other embodiments, the pharmaceutically acceptable carrier solution is a physiologic salt solution isotonic to blood serum. In some aspects of the present invention, the final concentrations of the pharmaceutical composition are 3 mg/L nonfunctionalized SWCNT and about 5 siRNA. In one or more embodiments, the pharmaceutical composition provides delivery of an effective amount of the siRNA and the effective amount reduces the expression of a target nucleic acid when compared to a strand of siRNA not complexed to the nonfunctionalized SWCNT. The actual dosage amount of a composition of the present invention administered to a patient or subject can be determined by physical and physiological factors such as body weight, severity of condition, the type of disease being treated, previous or concurrent therapeutic interventions, idiopathy of the patient and on the route of administration. The practitioner responsible for administration will, in any event, determine the concentration of SWCNT and/or bioactive substance in a composition and appropriate dose(s) for the individual subject.

In examples of some embodiments, pharmaceutical compositions may comprise, for example, at least about 0.1% of SWCNT complex. In other non-limiting examples, a dose may also comprise from about 1 microgram/kg/body weight, about 5 microgram/kg/body weight, about 10 microgram/kg/body weight, about 50 microgram/kg/body weight, about 100 microgram/kg/body weight, about 200 microgram/kg/body weight, about 350 microgram/kg/body weight, about 500 microgram/kg/body weight, about 1 milligram/kg/body weight, about 5 milligram/kg/body weight, about 10 milligram/kg/body weight, about 50 milligram/kg/body weight, about 100 milligram/kg/body weight, about 200 milligram/kg/body weight, about 350 milligram/kg/body weight, about 500 milligram/kg/body weight, to about 1000 mg/kg/body weight or more per administration, and any range derivable therein.

Various routes of administration are contemplated in aspects of the invention. In a particular embodiment, the SWCNT complexes are administered to a subject systemically. In other embodiments, methods of administration may include, but are not limited to, intravascular injection, intravenous injection, intraarterial injection, intratumoral injection, intraperitoneal injection, subcutaneous injection, intramuscular injection, transmucosal administration, oral administration, topical administration, local administration, or regional administration. In some embodiments, the complexes are administered intraoperatively. In other embodiments, the complexes are administered via a drug delivery device. According to other embodiments of the present invention, the SWCNT complexes necessitate only a single or very few treatment sessions to provide therapeutic treatment, which ultimately may facilitate patient compliance.

Some formulations are suitable for oral administration. Oral formulations include such typical excipients as, for example, pharmaceutical grades of mannitol, lactose, starch, magnesium stearate, sodium saccharine, cellulose, magnesium carbonate and the like.

Topical administration may be particularly advantageous for the treatment of skin cancers, to prevent chemotherapy-induced alopecia or other dermal hyperproliferative disorder. Such compositions would normally be administered as pharmaceutically acceptable compositions that include physiologically acceptable carriers, buffers or other excipients. For treatment of conditions of the lungs, or respiratory tract, aerosol delivery can be used. Volume of the aerosol is between about 0.01 ml and 0.5 ml.

An effective amount of the therapeutic composition is determined based on the intended goal. The term “unit dose” or “dosage” refers to physically discrete units suitable for use in a subject, each unit containing a predetermined-quantity of the therapeutic composition calculated to produce the desired responses discussed above in association with its administration, i.e., the appropriate route and treatment regimen. The quantity to be administered, both according to number of treatments and unit dose, depends on the protection or effect desired.

Precise amounts of the therapeutic composition also depend on the judgment of the practitioner and are peculiar to each individual. Factors affecting the dose include the physical and clinical state of the patient, the intended goal of treatment (e.g., alleviation of symptoms versus cure) and the potency, stability and toxicity of the particular therapeutic substance. The amount of SWCNT complexes administered to a patient may vary and may depend on the size, age, and health of the patient, the bioactive substance to be delivered, the indication being treated, and the location of diseased tissue. Moreover, the dosage may vary depending on the mode of administration.

### E. Combination Treatments

In certain embodiments, the SWCNT complexes may be administered to a subject in combination with one or more additional therapies.

The SWCNT complexes set forth herein may enhance the therapeutic or protective effect, and/or increase the therapeutic effect of another therapy. Therapeutic and prophylactic methods and compositions can be provided in a combined amount effective to achieve the desired effect. For example, if the disease is cancer, the therapeutic effect is the killing of a cancer cell and/or the inhibition of cellular hyperproliferation.

SWCNT complexes may be administered before, during, after or in various combinations relative to a secondary form of therapy. The administrations may be in intervals ranging from concurrently to minutes to days to weeks. In embodiments where the SWCNT complex is provided to a patient separately from the secondary therapeutic agent, one would generally ensure that a significant period of time did not expire between the time of each delivery, such that the two compounds would still be able to exert an advantageously combined effect on the patient. In such instances, it is contemplated that one may provide a patient with a SWCNT complex of the invention and the secondary therapy within about 12 to 24 or 72 h of each other or within about 6-12 h of each other. In some situations it may be desirable to extend the time period for treatment significantly where several days (2, 3, 4, 5, 6 or 7) to several weeks (1, 2, 3, 4, 5, 6, 7 or 8) lapse between respective administrations.

In certain embodiments, a course of treatment will last 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90 days or more. It is contemplated that one agent may be given on day 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, and/or 90, any combination thereof, and another agent is given on day 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, and/or 90, or any combination thereof. Within a single day (24-hour period), the patient may be given one or multiple administrations of the agent(s). Moreover, after a course of treatment, it is contemplated that there is a period of time at which no therapy is administered. This time period may last 1, 2, 3, 4, 5, 6, 7 days, and/or 1, 2, 3, 4, 5 weeks, and/or 1, 2, 3, 4, 5, 6, 7, 8, 9, 10, 11, 12 months or more, depending on the condition of the patient, such as their prognosis, strength, health, etc.

Various combinations may be employed. For the following non-limiting examples, the SWCNT complex therapy is “A” and an secondary therapy is “B”: AB/A; B/A/B; BIB/A; A/A/B; A/B/B; B/A/A; A/B/B/B; B/A/B/B; B/B/B/A; B/B/A/B; A/A/B/B; A/B/A/B; A/B/B/A; B/B/A/A; B/A/B/A; B/A/A/B; A/A/A/B; B/A/A/A; A/B/A/A; and A/A/B/A.

Administration of therapies of the present invention to a patient will follow general protocols for the administration of such compounds, taking into account the toxicity, if any, of the agents. Therefore, in some embodiments, there is a step of monitoring toxicity that is attributable to combination therapy. It is expected that the treatment cycles would be repeated as necessary.

In specific aspects, such as when the subject has a cancer, it is contemplated that combination therapy will include chemotherapy, radiotherapy, immunotherapy, surgical therapy or gene therapy in combination with the SWCNT complexes as set forth herein.

1. Chemotherapy

A wide variety of chemotherapeutic agents may be used in accordance with combination regimens of the present invention. The term “chemotherapy” refers to the use of drugs to treat cancer. A “chemotherapeutic agent” is used to connote a compound or composition that is administered in the treatment of cancer. These agents or drugs are categorized by their mode of activity within a cell, for example, whether and at what stage they affect the cell cycle. Most chemotherapeutic agents fall into the following categories: alkylating agents, antimetabolites, antitumor antibiotics, mitotic inhibitors and nitrosoureas.

Examples of chemotherapeutic agents include alkylating agents such as thiotepa and cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB 1-TM1); eleutherobin; pancratistatin; a sarcodictyin; spongistatin; nitrogen mustards such as chlorambucil, chlomaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gammall and calicheamicin omegall; dynemicin, including dynemicin A; bisphosphonates, such as clodronate; an esperamicm; as well as neocarzinostatin chromophore and related chromoprotein enediyne antiobiotic chromophores, aclacinomysins, actinomycin, authramycin, azasenne, bleomycins, cactinomycin, carabicin, caminomycin, carzinophilin, chromomycinis, dactinomycin, daunorubicin, detorubicin, 6-diazo-5-oxo-L-norleucine, doxorubicin (including morpholinodoxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, potfiromycin, puromycm, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; antiadrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK (polysaccharide complex); razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., paclitaxel and doxetaxel; chlorambucil; gemcitabine; 6-thioguanine; mercaptopurine; methotrexate; platinum coordination complexes such as cisplatin, oxaliplatin and carboplatin; vinblastine; platinum; etoposide (VP-16); ifosfamide; mitoxantrone; vincristine; vinorelbine; novantrone; teniposide; edatrexate; daunomycin; aminopterin; xeloda; ibandronate; irinotecan (e.g., CPT-11); topoisomerase inhibitor RFS 2000; difluoromethylomithine (DMFO); retinoids such as retinoic acid; capecitabine; cisplatin (CDDP), carboplatin, procarbazine, mechlorethamine, cyclophosphamide, camptothecin, ifosfamide, melphalan, chlorambucil, busulfan, nitrosurea, dactinomycin, daunorubicin, doxorubicin, bleomycin, plicomycin, mitomycin, etoposide (VP16), tamoxifen, raloxifene, estrogen receptor binding agents, taxol, paclitaxel, docetaxel, gemcitabien, navelbine, farnesyl-protein transferase inhibitors, transplatinum, 5-fluorouracil, vincristin, vinblastin and methotrexate and pharmaceutically acceptable salts, acids or derivatives of any of the above.

Also included in the definition of “chemotherapeutic agent” are antihormonal agents that act to regulate or inhibit hormone action on tumors such as antiestrogens and selective estrogen receptor modulators (SERMs), including, for example, tamoxifen, raloxifene, droloxifene, 4-hydroxytamoxifen, trioxifene, keoxifene, LY117018, onapristone, and toremifene; aromatase inhibitors that inhibit the enzyme aromatase, which regulates estrogen production in the adrenal glands, such as, for example, 4(5)-imidazoles, aminoglutethimide, megestrol acetate, exemestane, formestanie, fadrozole, vorozole, letrozole, and anastrozole; and anti-androgens such as flutamide, nilutamide, bicalutamide, leuprolide, and goserelin; as well as troxacitabine (a 1,3-dioxolane nucleoside cytosine analog); antisense oligonucleotides, particularly those which inhibit expression of genes in signaling pathways implicated in abherant cell proliferation, such as, for example, PKC-alpha, Ralf and H-Ras; ribozymes such as a VEGF expression inhibitor and a HER2 expression inhibitor; vaccines such as gene therapy vaccines and pharmaceutically acceptable salts, acids or derivatives of any of the above.

2. Radiotherapy

Other factors that cause DNA damage and have been used extensively include what are commonly known as y-rays, X-rays, and/or the directed delivery of radioisotopes to tumor cells. Other forms of DNA damaging factors are also contemplated such as microwaves, proton beam irradiation (E.g., U.S. Pat. Nos. 5,760,395 and 4,870,287) and UV-irradiation. It is most likely that all of these factors affect a broad range of damage on DNA, on the precursors of DNA, on the replication and repair of DNA, and on the assembly and maintenance of chromosomes. Dosage ranges for X-rays range from daily doses of 50 to 200 roentgens for prolonged periods of time (3 to 4 wk), to single doses of 2000 to 6000 roentgens. Dosage ranges for radioisotopes vary widely, and depend on the half-life of the isotope, the strength and type of radiation emitted, and the uptake by the neoplastic cells.

3. Immunotherapy

In the context of cancer treatment, immunotherapeutics, in general, rely on the use of immune effector cells and molecules to target and destroy cancer cells. Trastuzumab (Herceptin™) is such an example. The immune effector may be, for example, an antibody specific for some marker on the surface of a tumor cell. The antibody alone may serve as an effector of therapy or it may recruit other cells to actually affect cell killing. The antibody also may be conjugated to a drug or toxin (chemotherapeutic, radionuclide, ricin A chain, cholera toxin, pertussis toxin, etc.) and serve merely as a targeting agent. Alternatively, the effector may be a lymphocyte carrying a surface molecule that interacts, either directly or indirectly, with a tumor cell target. Various effector cells include cytotoxic T cells and NK cells. The combination of therapeutic modalities, i.e., direct cytotoxic activity and inhibition or reduction of ErbB2 would provide therapeutic benefit in the treatment of ErbB2 overexpressing cancers.

In one aspect of immunotherapy, the tumor cell must bear some marker that is amenable to targeting, i.e., is not present on the majority of other cells. Many tumor markers exist and any of these may be suitable for targeting in the context of the present invention. Common tumor markers include carcinoembryonic antigen, prostate specific antigen, urinary tumor associated antigen, fetal antigen, tyrosinase (P97), gp68, TAG-72, HMFG, Sialyl Lewis Antigen, MucA, MucB, PLAP, estrogen receptor, laminin receptor, erb B and p155. An alternative aspect of immunotherapy is to combine anticancer effects with immune stimulatory effects. Non-limiting examples of immune stimulating molecules include cytokines such as IL-2, IL-4, IL-12, GM-CSF, gamma-IFN, chemokines such as MIP-1, MCP-1, IL-8 and growth factors such as FLT3 ligand. Combining immune stimulating molecules, either as proteins or using gene delivery in combination with a tumor suppressor has been shown to enhance anti-tumor effects (Ju et al., 2000).

Non-limiting examples of immunotherapies include immune adjuvants e.g., Mycobacterium bovis, Plasmodium falciparum, dinitrochlorobenzene and aromatic compounds (U.S. Pat. Nos. 5,801,005 and 5,739,169; Hui and Hashimoto, 1998; Christodoulides et al., 1998), cytokine therapy, e.g., interferons α, β and γ; IL-1, GM-CSF and TNF (Bukowski et al., 1998; Davidson et al., 1998; Hellstrand et al., 1998) gene therapy, e.g., TNF, IL-1, IL-2, p53 (Qin et al., 1998; Austin-Ward and Villaseca, 1998; U.S. Pat. Nos. 5,830,880 and 5,846,945) and monoclonal antibodies, e.g., antiganglioside GM2, anti-HER-2, anti-p185 (Pietras et al., 1998; Hanibuchi et al., 1998; U.S. Pat. No. 5,824,311). It is contemplated that one or more anti-cancer therapies may be employed with the gene silencing therapies described herein.

In active immunotherapy, an antigenic peptide, polypeptide or protein, or an autologous or allogenic tumor cell composition or “vaccine” is administered, generally with a distinct bacterial adjuvant (Ravindranath and Morton, 1991; Morton et al., 1992; Mitchell et al., 1990; Mitchell et al., 1993).

In adoptive immunotherapy, the patient's circulating lymphocytes, or tumor infiltrated lymphocytes, are isolated in vitro, activated by lymphokines such as IL-2 or transduced with genes for tumor necrosis, and readministered (Rosenberg et al., 1989).

4. Surgery

Curative surgery is a cancer treatment that may be used in conjunction with the treatment of the present invention. Curative surgery includes resection in which all or part of cancerous tissue is physically removed, excised, and/or destroyed. Tumor resection refers to physical removal of at least part of a tumor. In addition to tumor resection, treatment by surgery includes laser surgery, cryosurgery, electrosurgery, and microscopically controlled surgery (Mohs' surgery). It is further contemplated that the present invention may be used in conjunction with removal of superficial cancers, precancers, or incidental amounts of normal tissue. Upon excision of part or all of cancerous cells, tissue, or tumor, a cavity may be formed in the body. Treatment may be accomplished by perfusion, direct injection or local application of the area with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months. These treatments may be of varying dosages as well.

5. Other Agents

It is contemplated that other agents may be used in combination with the present invention to improve the therapeutic efficacy of treatment. These additional agents include immunomodulatory agents, agents that affect the upregulation of cell surface receptors and GAP junctions, cytostatic and differentiation agents, inhibitors of cell adhesion, agents that increase the sensitivity of the hyperproliferative cells to apoptotic inducers, or other biological agents. Immunomodulatory agents include tumor necrosis factor; interferon alpha, beta, and gamma; IL-2 and other cytokines; F42K and other cytokine analogs; or MIP-1, MIP-1beta, MCP-1, RANTES, and other chemokines. It is further contemplated that the upregulation of cell surface receptors or their ligands such as Fas/Fas ligand, DR4 or DR5/TRAIL (Apo-2 ligand) would potentiate the apoptotic inducing abilities of the present invention by establishment of an autocrine or paracrine effect on hyperproliferative cells. Increases intercellular signaling by elevating the number of GAP junctions would increase the anti-hyperproliferative effects on the neighboring hyperproliferative cell population. In other embodiments, cytostatic or differentiation agents can be used in combination with the present invention to improve the anti-hyperproliferative efficacy of the treatments. Inhibitors of cell adhesion are contemplated to improve the efficacy of the present invention. Examples of cell adhesion inhibitors are focal adhesion kinase (FAKs) inhibitors and Lovastatin. It is further contemplated that other agents that increase the sensitivity of a hyperproliferative cell to apoptosis, such as the antibody c225, could be used in combination with the present invention to improve the treatment efficacy.

### F. Kits and Diagnostics

In various aspects of the invention, a kit is envisioned containing SWCNT complexes as set forth herein. In some embodiments, the present invention contemplates a kit for preparing and/or administering a SWCNT complex of the present invention. The kit may comprise one or more sealed vials containing any of the SWCNT complexes set forth herein or reagents for preparing any of the SWCNT complexes set forth herein. In some embodiments, the kit may also comprise a suitable container means, which is a container that will not react with components of the kit, such as an eppendorf tube, an assay plate, a syringe, a bottle, or a tube. The container may be made from sterilizable materials such as plastic or glass.

The kit may further include an instruction sheet that outlines the procedural steps of the methods, and will follow substantially the same procedures as described herein or are known to those of ordinary skill. The instruction information may be in a computer readable media containing machine-readable instructions that, when executed using a computer, cause the display of a real or virtual procedure of delivering a pharmaceutically effective amount of the SWCNT complexes of the present invention.

### EXAMPLES

In order that the invention disclosed herein may be more efficiently understood, examples are provided. The following examples are for illustrative purposes only and are not to be construed as limiting the invention in any manner.

### Example 1.0

### Materials and Methods

### Example 1.1

### Preparation of Noncovalent Complexes of SWCNTs with siRNA

SWCNTs were produced using a high-pressure carbon monoxide (HiPco) process. The raw HiPco SWCNT product was added to an aqueous buffer solution (100 mM KCl, 30 mM HEPES-KOH [pH 7.5], 1 mM MgCl₂) containing 20 μM solubilized pooled siRNA [(siRNA targeting HIF-1α (HIF-1α) 5′-CCUGUGUCUAAAUCUGAAC-3′ (SEQ ID NO:6), 5′CUAC CUUCGUGAUUCUGUUU-3′(SEQ ID NO:7), GCACAAUAGACAGCGAAAC-3′ (SEQ ID NO:8), 5′-CUACUUUCUUAA UGGCUUA (SEQ ID NO:9), polo-like kinase 1 (PLK1), 5′-CAACCAAAGUCG AAUAUUGAUU-3 (SEQ ID NO:10), 5′-C AAGAAGAAUGAAUACAGUUU-3′ (SEQ ID NO:11), 5′-GAAGAUGUCCAUGGAAAUAUU-3′ (SEQ ID NO:12), 5′-CAACA CGCCUCAUCCUCUAUU-3′ (SEQ ID NO:13), Kinesin superfamily protein (Kif11), 5′-CGUCUUUAGAU UCCUAUAU-3′ (SEQ ID NO:14), 5′GUUGUUCCUACUUCAGAUA-3′ (SEQ ID NO:15), 5′-GUCGUCUUUAGAUUCCUAU-3′ (SEQ ID NO:16), 5′-GAUCUACCGAAAGAGUCAU-3′ (SEQ ID NO:17), non-targeting siRNA 5′-UAGCGACAUU UGUGUAGUU-3′ (SEQ ID NO:18) or siTox, purchased from Dharmacon Inc, IL. This mixture was sonicated (Sonics, Vibra-cell) at 25° C. using two 15 second pulses at settings of 130 W, 20 k Hz, and 40% amplitude. The sonicated sample was centrifuged at 15,000×g for 5 minutes. The pellet comprising bundled SWCNTs was discarded and the supernatant was transferred into a clean tube and centrifuged an additional 1 minute at the same settings. The resulting supernatant contained SWCNTs noncovalently suspended by coatings of adsorbed siRNA. Near infrared (NIR) fluorescence spectroscopy indicated that the sample contained predominantly individually suspended SWCNTs rather than nanotube aggregates.

### Example 1.2

### Stability and Biological Activity

The SWCNT/siRNA complexes were stable and retained their biological activity following 30 days of storage at 4° C. It is predicted that the SWCNT/siRNA complexes could retain biological activity following longer periods of storage at 4° C.

### Example 1.3

### Cell Culture and Cellular Incubation with SWCNT/siRNA Complexes

MiaPaCa2-HRE (a pancreatic cell line with a HIF-1α/luciferase reporter) cells were incubated in growth media consisting of high glucose DMEM supplemented with 10% fetal calf serum (FCS) (all reagents from HyCone). To determine the internalization rate of non-targeting siRNA-solubilized SWCNTs, 50 μL. of the complex (final SWCNT concentration approximately 1.25 mg/L) was added to cells (approximately 2×10⁵ cells/well) that had been incubated for 18 hours in 1 mL of media in a 6-well plate. Incubation with the SWCNT/siRNA complex continued for 1, 3 and 6 hours. After incubation, media was removed from the wells, the cells were washed once in phosphate buffered saline (PBS) and then were detached from the surface by adding 0.25% trypsin (Invitrogen). The detached cells were washed with growth media to inactivate the trypsin and then washed again with PBS. The cells were resuspended in 1 mL of growth media, transferred onto a circular glass cover slip in a well of a new 6-well plate and incubated at 37° C. in a humid environment for approximately 20 hours. NIR fluorescence microscopy was utilized to identify internalized SWCNTs.

To investigate the biological activities of SWCNT/siRNA complexes, 20 μL of each sample was added to cells (approximately 2×10⁵ cells/well) in 100 μL of media containing 10% FCS in 96-well plates: The plates were incubated at 37° C. in a humidified chamber for approximately 18 hours prior to and for 72 hours following addition of the complexes. To determine the ability of the complexes to suppress HIF-1α activity or silence the HIF-1α protein, treated cells incubated under normoxia for 72 hours were incubated for a further 18 hours under hypoxic conditions (1% oxygen).

### Example 1.4

### Cell Viability

Cell proliferation reagent (WST-1, Roche, Mannheim Germany) was added to cells in media to a final concentration of 10% and the cells were incubated for minutes at 37° C. in a humidified incubator. The absorbance of the sample was then measured relative to a background control using a microplate reader (Polar Star Optima; BMG Labtech) at 420-480 nm.

### Example 1.5

### Reporter Assay

The MiaPaCa2-HRE cell line was generated to stably express the promoter sequence of a target gene of HIF-1α comprising the HIF-1α binding hypoxia response element (HRE) fused to the luciferase gene. At the end of the experiment, 100 μL of media was removed from each well of the 96-well plate. The removed media was replaced with 50 μL of the luciferase reagent (25 mM tricine, 0.5 mM EDTA-Na₂, 0.54 mM sodium triphosphate, 16.3 mM MgSO₄.7H₂O, 0.3% Triton X-100, 0.1% w/v dithiothreitol, 1.2 mM ATP, 50 mM luciferin, and 270 mM coenzyme A). The plates were incubated at room temperature for 5 minutes. Sample luminescence was measured relative to a background control using a microplate reader (Polar Star Optima; BMG Labtech).

### Example 1.6

### Spectroscopy and Microscopy Characterization of SWCNTs

The NIR emission spectrum of the siRNA-suspended SWCNTs was measured using 658 nm excitation in a model NSI NanoSpectralyzer (Applied NanoFluorescence, Houston, Tex.). NIR fluorescence microscopy was performed using a custom-built apparatus containing diode laser excitation sources emitting at 658 and 785 nm. Individual SWCNTs internalized into cells were imaged with a custom-built NIR fluorescence microscope using 785 nm excitation, a 60× oil-immersion objective, and a 946 nm long-pass filter in the collection path. Bright field images were taken using the X objective.

### Example 1.7

### Statistical Analysis

Statistical analyses were performed with commercially available software. Single regression analysis was used to assess the ratio of HIF-1 activity after treatment with 100 μL sample volume, SWCNT concentration approximately 4 mg/L, siRNA concentration approximately 2 μM, with the percentage luciferase expression after SWCNT/siRNA treatment as the dependent variable. Student's t-tests were used to compare the ratio of luciferase intensity within the tumor between mice treated with SWCNT/siRNA. Comparisons of mice treated with siRNA targeting HIF-1 (siHIF), SWCNT/non-targeting siRNA (SWCNT/SC), or SWCNT/siRNA targeting HIF-1α were computed by two-way analysis of variance (ANOVA). Statistical significance was defined as a P value of <0.05.

### Example 2.0

### Animal Studies

### Example 2.1

### Testing the Biological Activity of the siRNAISWCNT Complexes in 0.9% Saline Solution

SWCNTs were complexed with 20 μM of siRNA targeting polo-like kinase1 (PLK1) in a 0.9% NaCl solution using the procedure described above. A 20 μL portion of each sample was added to cells (approximately 2×10⁵ cells/well) in 100 μL of media containing 10% FCS in 96-well plates. The treated cells were incubated at 37° C. in a humid chamber for 72 hours and their viability was determined by the WST-1 assay.

### Example 2.2

### Injection of Mice with MiaPaCa-2/HRE Pancreatic Cancer Cells

The cells were grown in humidified 95% air, 5% CO₂ at 37° C. in DMEM supplemented with 10% FCS. Cells (10⁷) in log cell growth were suspended in 0.1 mL Matrigel (Becton Dickinson Biosciences, Palo Alto, Calif.) and subcutaneously injected into the flanks of female Swiss nu/nu mice (Charles River laboratories, Wilmington, Mass.). Tumor diameters at right angles (dₛₕₒᵣₜ and d_{long}) were measured twice weekly with electronic calipers and converted to volume by the formula: volume=dₛₕₒᵣₜ²×d_{long}/2. When the tumors reached 150 mm³, the mice were stratified into groups of 8 animals having approximately equal mean tumor volumes. Intra-tumoral administration of the siRNA/SWCNT complexes was then performed twice per week for 3 weeks (100 μL sample volume, SWCNT concentration approximately 4 mg/L, siRNA concentration approximately 2 μM). The intra-tumoral injections were administered with the mice positioned dorsally and their tumors divided into four quadrants. Each injection was administered in a new quadrant using a clockwise rotation. Tumor volume was measured twice weekly until the tumor reached 1500 mm³ or more or became necrotic, at which time the mice were euthanized.

### Example 2.3

### Detecting Luciferase Expression In Vivo

### Bioluminescence Imaging

After 20 days of tumor development, mice were imaged twice weekly using the IVIS Lumina (Caliper Life Sciences). Mice were pair-matched into groups according to their tumor volumes. Before imaging, D-Luciferin (Caliper Life Sciences) was given to each mouse via intraperitoneal injection at a dose of 150 mg/kg and allowed to distribute for 5 minutes. The mice were anesthetized in the chamber with 3% isoflurane and then imaged using a 12.5 cm field of view and a 15 second exposure time. Their respective bioluminescence intensities were determined by calculating the photon flux using Living Image software (version 3.0). Photon flux was represented as photons/s/cm²/sr in the region of interest (ROI) and surrounding bioluminescence signal provided by the tumor. The ROIs were then used to determine the photon flux, expressed as percent photon flux of vehicle control values.

### Example 2.4

### Western Blotting

Cell pellets were resuspended in modified RIPA lysis buffer (10 mM NaCl, 1% NP-40, 0.5% sodium deoxycholate, 0.1% SDS, 50 mM tris-hydrochloric acid [pH 7.5] with inhibitors (20 μg/mL aprotinin, 1 mM sodium fluoride, 2 mM sodium orthovanadate, 0.5 mM phenylmethanesulfonyl fluoride, and 250 mg/mL benzamidine) in ice for 30 minutes and centrifuged at 15 000×g for 30 minutes to collect whole cell lysates. The lysates (50-60 μg) were run on 10% SDS-polyacrylamide electrophoresis (PAGE) gels and transferred to a polyvinylidene difluoride membrane. Western blotting was performed with specific primary antibodies and peroxidase-conjugated affiniPure anti-Mouse and anti-Rabbit secondary antibodies (Jackson ImmunoResearch Laboratories). Proteins were visualized with ECL Plus enhanced chemiluminescence reagents (Amersham Biosciences, Piscataway, N.J.).

### Example 3.0

### Results

### Example 3.1

### siRNA Suspends Pristine SWCNTs

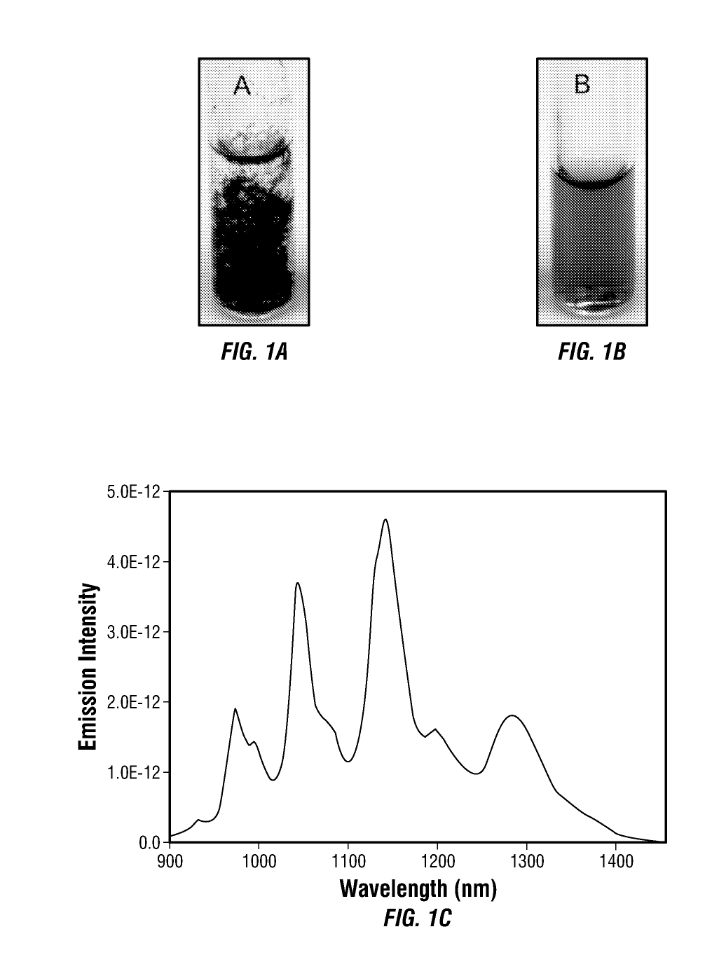

The unagglomerated, nonfunctionalized SWCNTs are made water-compatible by coating with siRNA. As shown in FIG. 1A, sonication of nonfunctionalized SWCNTs in aqueous buffer in the absence of siRNA failed to produce a stable suspension. However, as shown in FIG. 1B, equivalent processing in the presence of siRNA provided stable, homogeneous suspensions. These suspensions displayed strong NIR fluorescence between approximately 900 and 1600 nm, as depicted in FIG. 1C, which is characteristic of dispersed or unagglomerated SWCNTs.

### Example 3.2

### siRNA-Solubilized Nonfunctionalized SWCNTs Rapidly Internalized Into Pancreatic Cancer Cells

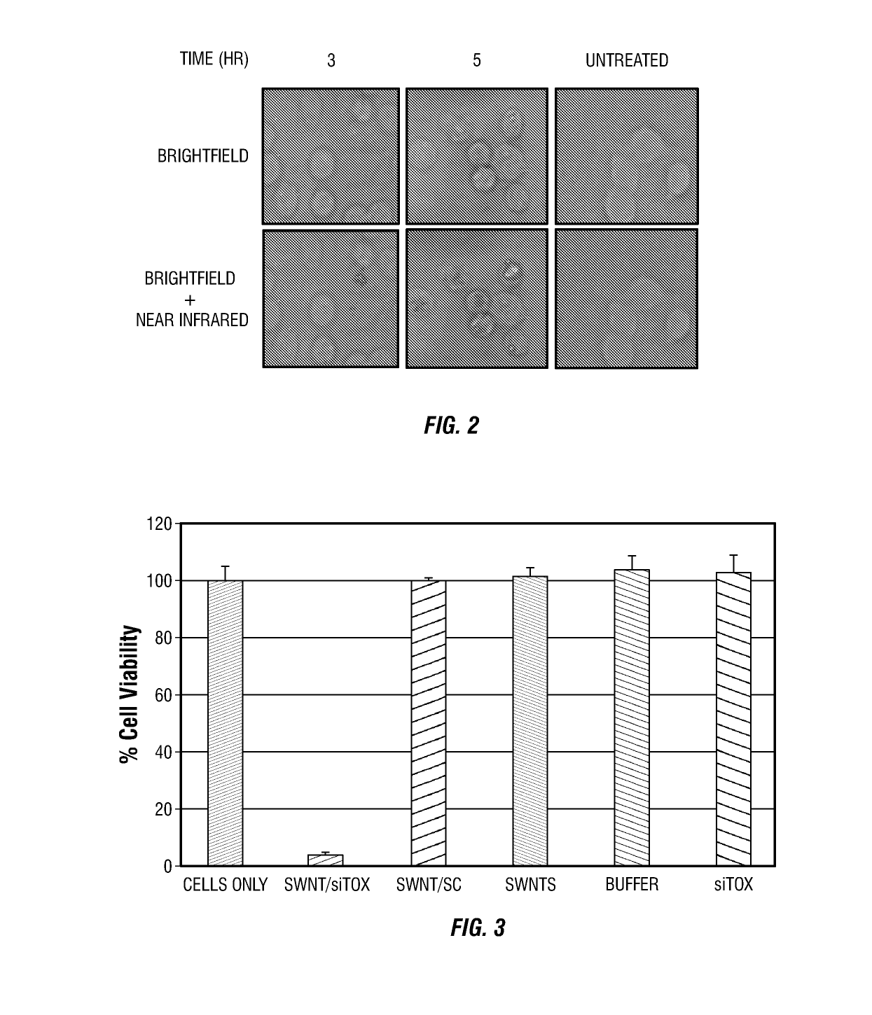

MiaPaCa2-HRE cultures were exposed to SWCNT/siRNA complexes for 1, 3 and 6 hours to monitor internalization of the complex into tissue cells. As shown in FIG. 2, NIR fluorescence microscopy of the treated cells revealed internalized SWCNTs. The cells having internalized SWCNTs were characterized by their emission wavelengths and their strong dependence of emission intensity on excitation beam polarization. In addition, NIR fluorescent particles were found only in cells incubated with suspended SWCNTs and not in SWCNT-free control samples. As the sample area irradiated by the laser beam was smaller than the image field, some cells in each image did not show NIR emission even though they contain internalized SWCNTs. Incubation with the SWCNT/siRNA complexes for 1 hour resulted in SWCNT uptake by approximately 40% of cells. Incubation for 3 hours or 6 hours resulted in nanotube uptake by larger fractions of cells, and average SWCNT content per cell also increased with incubation time. Although the concentration of internalized nanotubes varied substantially from cell to cell, after 6 hours of incubation, more than 90% of the cells showed detectable SWCNTs.

### Example 3.3

### Internalized SWCNTs Deliver siRNA Capable of Inducing a Biological Response

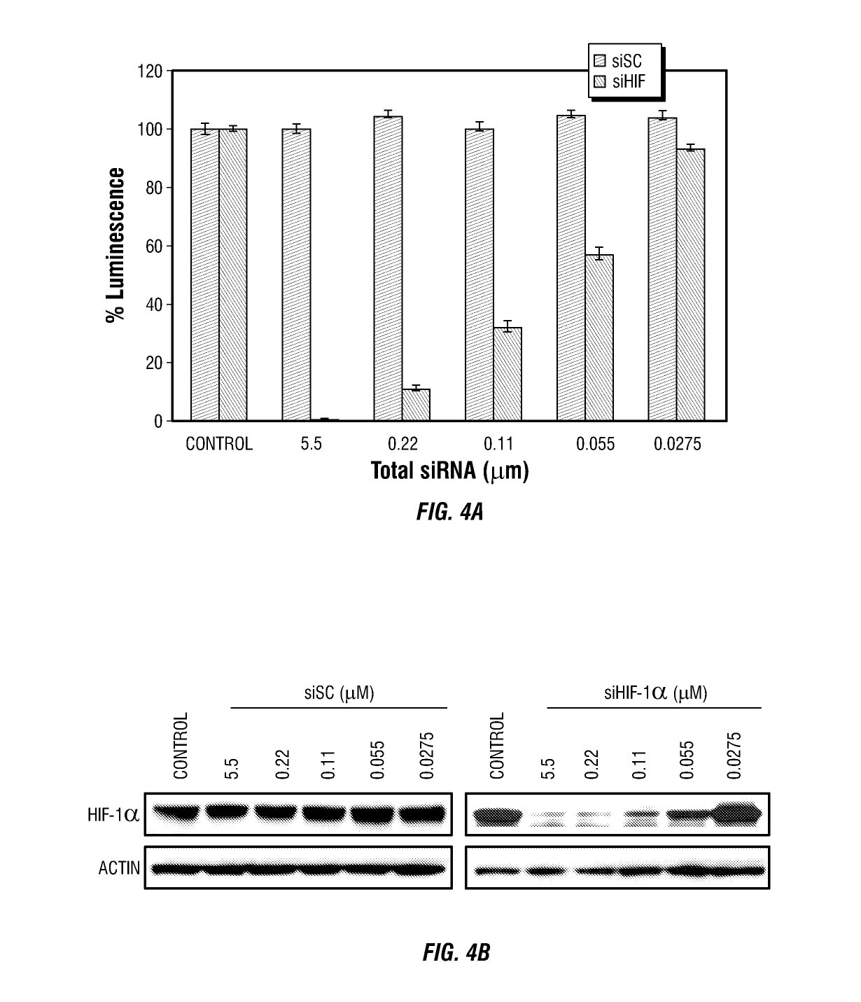

A mixture of pristine SWCNTs and siTox was sonicated and 20 μL of the complex (containing 5 mg/L SWCNTs and 5 μM siTox) was added to MiaPaCa-HRE (human pancreatic cancer) cells growing in a 96-well plate. Each well contained 100 μl, of medium with 10% FCS. Controls included untreated cells and cells treated with 20 mL of a complex of SWCNT and non-targeting siRNA (SWCNTISC) (containing 5 mg/L SWCNTs and 5 μM siSC), 20 μL of SWCNTs solubilized by 10% FCS, buffer alone and free uncomplexed siTox (final concentration 5 μM). At 72 hours after treatment, a decrease of approximately 90% was observed in viability of cells treated with the SWCNT/siTox complex, as shown in FIG. 3. This effect was specific to the SWCNT/siTox complex, as none of the controls exhibited decreased cell viability. The preparative sonication did not damage the siRNA and siRNA was delivered into cells in a biologically active form. Further, the presence of serum did not inhibit the transfection process.

### Example 3.4

### siRNA Delivered into Cells by Nonfunctionalized SWCNTs Induces RNAi Response

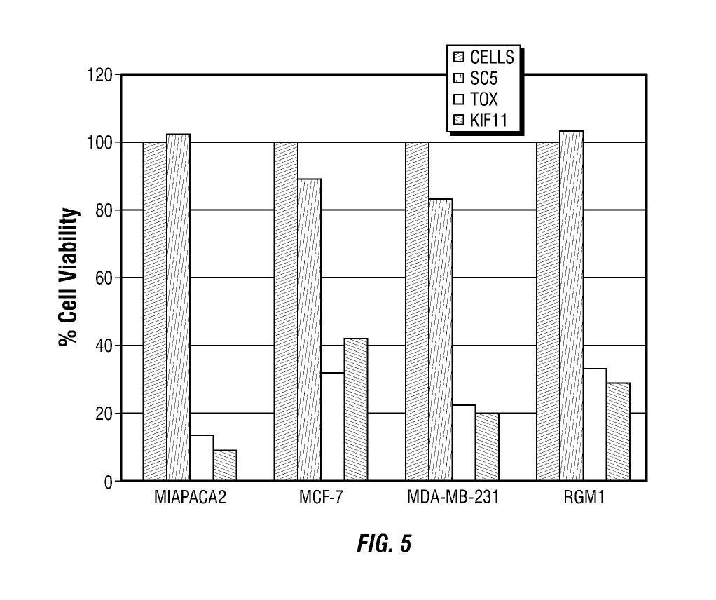

It was investigated whether SWCNT/siRNA complexes could activate a specific RNAi response. The model for the experiment was the MiaPaCa-HRE pancreatic cancer cell line. Changes in HIF-1α activity were monitored in these cells by measuring the levels of luciferase expression. MiaPaCa-HRE cells were treated with SWCNTs complexed with either an siRNA specifically targeting HIF-1α (siHIF), or a non-targeting siRNA (siSC), at final concentrations of 3 mg/L SWCNTs and 5 μM siRNA. The final siRNA concentration was based on the initial siRNA concentration suspended in the siRNA buffer and, as such, the final siRNA concentration likely exceeded the actual concentration of siRNA complexed to SWCNTs and the actual concentration taken into cells by SWCNTs. Treated cells were incubated under normoxic conditions at 37° C. for 72 hours and then were transferred into a hypoxic chamber (1% oxygen) for an additional 18 hours. HIF-1 activity was found to be significantly inhibited in cells treated with the SWCNT-siHIF-1α complex, but unchanged in cells treated with the SWCNT/siSC complex, as shown in FIG. 4A. Western blotting, as shown in FIG. 4B, confirmed that the inhibition of HIF-1 activity was the result of knockdown of the protein. The loss of HIF-1 activity and protein knockdown correlated well in a concentration-dependent manner. Because knockdown of the HIF-1α protein was observed only in cells treated with SWCNT/siHIF-1α complexes, it is likely that siRNAs retain their ability to induce a specific RNAi response after delivery into cells by complexation with nonfunctionalized SWCNTs.

### Example 3.5

### SWCNT/siRNA Complexes Effectively Induce RNAi Response in Multiple Cell Types

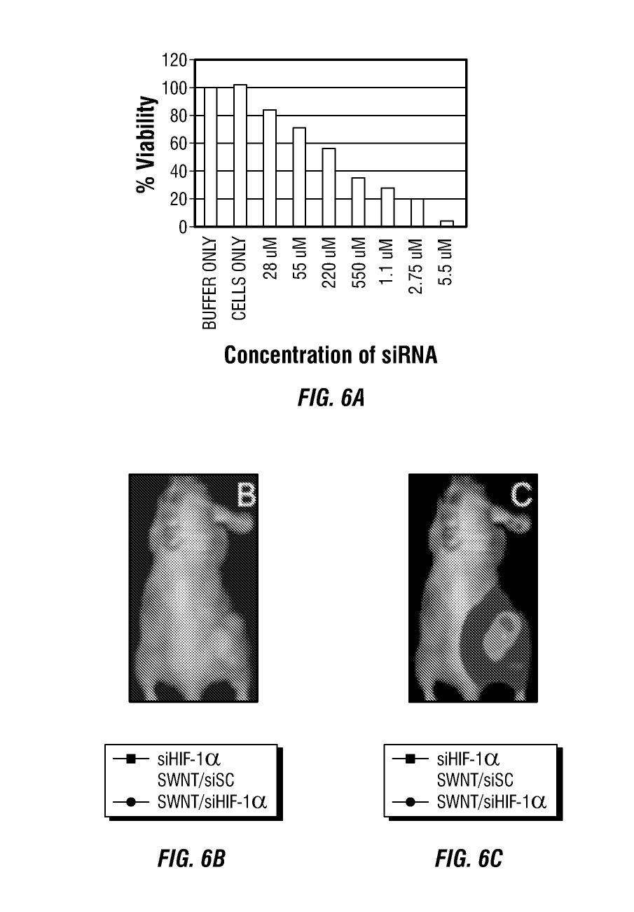

Complexes of either SWCNT/non-targeting siRNA (siSC), SWCNT/siRNA targeting Kif11 (siKif11) or SWCNT/siRNA Tox (siTox) at a final concentration of 5 mM were added to cells growing in normal media containing 10% FCS. SWCNT/siRNA complexes were added to cultures of pancreatic cancer cells (MiaPaCa2), breast cancer cells (MCF-7, MDA-MB-231), and ovarian cancer cell line (RGM1) to determine if SWCNTs could deliver siRNA into a wide range of cell types to induce the RNAi response. Cells were incubated at 37° C. for 72 h. Cell viability was determined by the WST-1 Assay. As shown in FIG. 5, non-targeting siRNA (siSC) demonstrated negligible toxicity to the cancer cells tested while siTox and siKif11 both induced cell death in transfected cells. These results suggest that SWCNTs have the potential to function as a serum-insensitive, wide range transfection agent to deliver siRNA into cancer cells to induce the RNAi response.

### Example 3.6

### Intratumoral Administration of SWCNT/siRNA Complexes inhibits HIF-1α Activity in a Xenograft Mouse Tumor

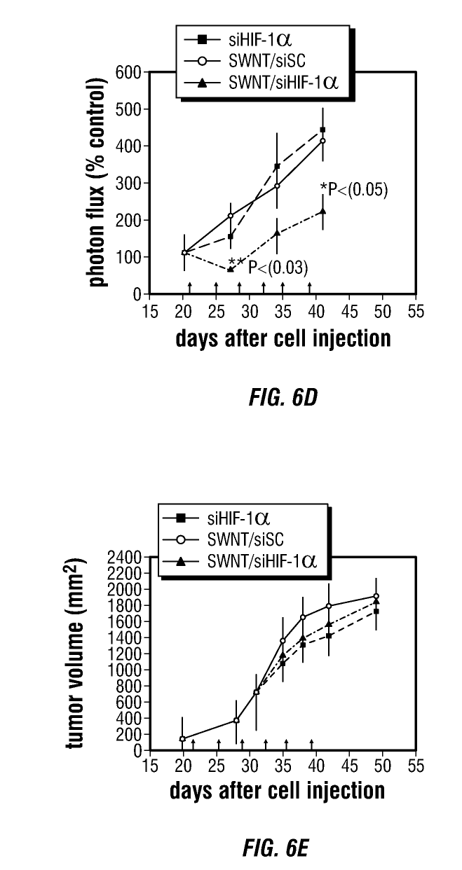

FIGS. 6A-6E illustrate the inhibition of HIF-1α activity in a xenograft mouse tumor after administration of SWCNT/siRNA complexes. In particular, the xenograft mouse tumor model was utilized to investigate the ability of SWCNT/siHIF complexes to inhibit HIF-1α activity in vivo. An 0.9% saline solution was utilized as an alternative to the siRNA buffer. In order to demonstrate that a similar biological outcome using siRNA/SWCNTs complexes in 0.9% saline can be achieved, complexes in saline were prepared at several concentrations, as described for the siRNA buffer and added to MiaPaCa-HRE pancreatic cancer cells growing in normal media containing 10% FCS. siRNA targeting Polo-like Kinase 1 (PLK1), a protein that plays an important role in the G2-M transition and whose silencing results in cell death, was utilized. As shown in FIG. 6A, the saline environment provided no significant change in biological activity of the SWCNT/siRNA complexes at concentrations used for the animal study.

To study the effectiveness of targeting MiaPaCa-HRE cells in vivo, cell suspensions were subcutaneously injected into the right flanks of 6 to 8-week-old female athymic nude mice (nu/nu). Activation of HIF-1α in the hypoxic environment of the growing tumor was confirmed by imaging the bioluminescence of luciferin. Because MiaPaCa cell lines do not express Hif-2a, the images allowed HIF-1α activity to be monitored in vivo in the xenograft mouse model, as depicted in FIGS. 6B and 6C. Significantly decreased tumor HIF-1α activity was observed in mice treated with SWCNT/HIF complexes compared to those treated with complexes comprising either the control SWCNT/siRNA (p<0.01 to p<0.05) or HIF-1α siRNA alone (FIG. 6D). However, no suppression of tumor volume was observed (FIG. 6E), a result possibly attributable to incomplete inhibition of HIF-1α. To test this possibility, an ex-vivo experiment was conducted in which MiaPaCa-HRE parental cells, cells transfected with a control siRNA/SWCNT complex, and siHIF/SWCNT complex were grown in tissue culture for 24 hours prior to being injected subcutaneously into mice. Tumor growth was monitored over a period of 33 days. It was observed that tumors generated by the parental cells and those transfected with the control siRNA grew similarly and at a faster rate compared to tumors transfected with the siRNA targeting HIF-1α. An initial period of growth inhibition of the tumors transfected with the siRNA targeting HIF-1α accounted for the slow rate of growth compared to the other two groups. No significant difference in the levels of HIF-1α was observed between the three groups. This may be due at least in part because protein silencing by siRNA is a transient effect, usually lasting up to about one week.

Transfecting cells for periods longer than 6 hours with SWCNT/siRNA results in both a significant uptake of the complexes into the cells, as shown in FIG. 2, and silencing of HIF-1α expression, as shown in FIG. 4B. As such, the initial growth inhibition observed in our ex-vivo study was most probably due to the complete inhibition of HIF-1α.

### Example 3.7

### Toxicity

Even at high concentrations, toxicity was not observed following intravenous administration of either nonfunctionalized SWCNTs or coated SWCNTs of the present invention. No mortality or loss of weight of mice as well as no evidence of toxicity in tissues and organs were observed in these studies that ranged in time from 24 hours to 6 months after treatment.

### Example 3.8

### Summary of Results

The results demonstrate that siRNA can be used to solubilize nonfunctionalized SWCNTs and that noncovalent SWCNT/siRNA complexes can transfect cancer cells and effectively silence a targeted gene in cell culture and also in tumors in vivo. In addition, siRNA can be used to silence target genes with a high degree of specificity. The results further demonstrate that numerous siRNA sequences can be utilized to complex the nonfunctionalized SWCNTs and that irrespective of their nucleotide sequences, the siRNA solubilized the SWCNTs equally effectively. This observation differs from observations that the ability of single stranded DNA to solubilize nonfunctionalized SWCNTs is dependent on the guanine-cytosine (GC) content of the nucleotide sequence.

Efficient intracellular transport and delivery of siRNA is critical to the potency and in vivo therapeutic activity of RNAi. Internalization of the SWCNT/siRNA complex was observed in about 30% of the treated cells 1 hour after addition of the complex to cells growing in media containing 10% serum. By 3 hours post treatment, internalized SWCNTs were observed in more than 90% of cells and the number of internalized SWCNTs per cell increased further by 6 hours.

There are significant differences between SWCNTs and lipid reagents as delivery agents of siRNA. Commercial lipid reagents are cell line specific and to obtain optimum transfection conditions with minimum toxicity requires selecting the best reagent from a panel of lipid reagents. The SWCNTs are much less cell line dependent and have negligible toxic effects on most cell lines. In addition, lipid reagent transfections generally have to be carried out in the absence of serum, which is toxic to cells. Conversely, SWCNTs transfections of the present invention can be carried out in the presence of serum.

The sonication protocol for forming SWCNT/siRNA complexes does not functionally damage the siRNA, as cells exposed to the complexes display a clear RNAi response. Both HIF-1α activity and protein levels were lowered by approximately 70% to 80% when the nonfunctionalized SWCNTs delivered siRNA targeting HIF-1α mRNA into the host cancer cells.

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and methods described herein without departing from the concept, spirit and scope of the invention. Such variations are intended to fall within the scope of the appended claims.

What is claimed is:

1. A single-walled carbon nanotube composition for delivery of siRNA comprising:
a) a nonfunctionalized single-walled carbon nanotube; and
b) siRNA noncovalently complexed with the nonfunctionalized single-walled carbon nanotube, wherein the siRNA solubilizes such nonfunctionalized single-walled carbon nanotube.

2. The single-walled carbon nanotube composition of claim 1, wherein the nonfunctionalized single-walled carbon nanotube is unagglomerated and nonaggregated.

3. The single-walled carbon nanotube composition of claim 1, wherein the diameter of the nonfunctionalized single-walled carbon nanotube is about 1 nm to about 2 nm.

4. The single-walled carbon nanotube composition of claim 1, wherein the diameter of the nonfunctionalized single-walled carbon nanotube is about 1 nm.

5. The single-walled carbon nanotube composition of claim 1, wherein the length of the nonfunctionalized single-walled carbon nanotube is about 500 nm or less.

6. The single-walled carbon nanotube composition of claim 1, wherein the length of the nonfunctionalized single-walled carbon nanotube is about 400 nm or less.

7. The single-walled carbon nanotube composition of claim 1, wherein the length of the nonfunctionalized single-walled carbon nanotube is about 100 nm to about 300 nm.

8. The single-walled carbon nanotube composition of claim 1, wherein the length of the nonfunctionalized single-walled carbon nanotube is about 125 nm to about 275 nm.

9. The single-walled carbon nanotube composition of claim 1, wherein the length of the nonfunctionalized single-walled carbon nanotube is about 150 nm to about 250 nm.

10. The single-walled carbon nanotube composition of claim 1, wherein the length of the nonfunctionalized single-walled carbon nanotube is about 175 nm to about 225 nm.

11. The single-walled carbon nanotube composition of claim 1, wherein the siRNA comprises chemically-modified siRNA.

12. The single-walled carbon nanotube composition of claim 1, wherein the siRNA comprises stabilized siRNA.

13. The single-walled carbon nanotube composition of claim 1, wherein the siRNA comprises non-targeting siRNA.

14. The single-walled carbon nanotube composition of claim 1, wherein the siRNA comprises targeting siRNA.

15. The single-walled carbon nanotube composition of claim 14, wherein the siRNA is targeted to hypoxia-inducible factor 1 alpha (HIF-1α) mRNA.

16. The single-walled carbon nanotube composition of claim 14, wherein the siRNA is targeted to vascular endothelial growth factor (VEGF) mRNA.

17. The single-walled carbon nanotube composition of claim 16, wherein the sense strand of the siRNA is AUGUGAAUGCAGACCAAAGAA (SEQ ID NO: 1).

18. The single-walled carbon nanotube composition of claim 14, wherein the siRNA is targeted to endothelial growth factor receptor (EGFR) mRNA.

19. The single-walled carbon nanotube composition of claim 18, wherein the sense strand of the siRNA is GUCAGCCUGAACAUAACAU (SEQ ID NO: 2).

20. The single-walled carbon nanotube composition of claim 18, wherein the sense strand of the siRNA is GUGUAACGGAAUAGGUAUU (SEQ ID NO: 3).

21. The single-walled carbon nanotube composition of claim 14, wherein the siRNA is targeted to human epidermal growth factor receptor 2 (HER2) mRNA.

22. The single-walled carbon nanotube composition of claim 21, wherein the sense strand of the siRNA is GGAGCUGGCGGCCUUGUGCCG (SEQ ID NO: 4).

23. The single-walled carbon nanotube composition of claim 21, wherein the sense strand of the siRNA is UCACAGGGGCCUCCCCAGGAG (SEQ ID NO: 5).

24. A single-walled carbon nanotube composition comprising a nonfunctionalized single-walled carbon nanotube and a siRNA noncovalently solubilizing such nonfunctionalized single-walled carbon nanotube, wherein the single-walled carbon nanotube composition is internalized in treated cells in media containing serum at a rate measured in vitro that substantially corresponds to the following:
(i) from about 0.01% to about 30% of the total amount of treated cells internalize the single-walled carbon nanotube composition after about 1 hour of measurement;
(ii) from about 20% to about 90% of the total amount of treated cells internalize the single-walled carbon nanotube composition after about 3 hours of measurement; and
(iii) not less than about 95% of the total amount of treated cells internalize the single-walled carbon nanotube composition after about 24 hours of measurement.

25. The single-walled carbon nanotube composition of claim 24, wherein the siRNA dissociates from the single-walled carbon nanotube when internalized in the treated cell.

26. The single-walled carbon nanotube composition of claim 24, wherein the siRNA remains complexed with the single-walled carbon nanotube when internalized in the treated cell.

27. A pharmaceutical composition comprising:
a) a nonfunctionalized single-walled carbon nanotube;
b) an siRNA noncovalently complexed with the nonfunctionalized single-walled carbon nanotube; and
c) a pharmaceutically acceptable carrier, wherein such nonfunctionalized single-walled carbon nanotube is solubilized into the pharmaceutically acceptable carrier by association with such siRNA.

28. The pharmaceutical composition of claim 27, wherein the nonfunctionalized single-walled carbon nanotube is unagglomerated and nonaggregated.

29. The pharmaceutical composition of claim 27, wherein the diameter of the nonfunctionalized single-walled carbon nanotube is about 1 nm to about 2 nm.

30. The pharmaceutical composition of claim 27, wherein the diameter of the nonfunctionalized single-walled carbon nanotube is about 1 nm.

31. The pharmaceutical composition of claim 27, wherein the length of the nonfunctionalized single-walled carbon nanotube is about 500 nm or less.

32. The pharmaceutical composition of claim 27, wherein the length of the nonfunctionalized single-walled carbon nanotube is about 400 nm or less.

33. The pharmaceutical composition of claim 27, wherein the length of the nonfunctionalized single-walled carbon nanotube is about 100 nm to about 300 nm.

34. The pharmaceutical composition of claim 27, wherein the length of the nonfunctionalized single-walled carbon nanotube is about 125 nm to about 275 nm.

35. The pharmaceutical composition of claim 27, wherein the length of the nonfunctionalized single-walled carbon nanotube is about 150 nm to about 250 nm.

36. The pharmaceutical composition of claim 27, wherein the length of the nonfunctionalized single-walled carbon nanotube is about 175 nm to about 225 nm.

37. The pharmaceutical composition of claim 27, wherein the siRNA comprises chemically modified siRNA.

38. The pharmaceutical composition of claim 27, wherein the siRNA comprises stabilized siRNA.

39. The pharmaceutical composition of claim 27, wherein the siRNA comprises nontargeting siRNA.

40. The pharmaceutical composition of claim 27, wherein the siRNA comprises targeting siRNA.

41. The pharmaceutical composition of claim 40, wherein the siRNA is targeted to hypoxia-inducible factor 1 alpha (HIF-1α) mRNA.

42. The pharmaceutical composition of claim 40, wherein the siRNA is targeted to vascular endothelial growth factor (VEGF) mRNA.

43. The pharmaceutical composition of claim 42, wherein the sense strand of the siRNA is AUGUGAAUGCAGACCAAAGAA (SEQ ID NO: 1).

44. The pharmaceutical composition of claim 40, wherein the siRNA is targeted to endothelial growth factor receptor (EGFR) mRNA.

45. The pharmaceutical composition of claim 44, wherein the sense strand of the siRNA is GUCAGCCUGAACAUAACAU (SEQ ID NO: 2).

46. The pharmaceutical composition of claim 44, wherein the sense strand of the siRNA is GUGUAACGGAAUAGGUAUU (SEQ ID NO: 3).

47. The pharmaceutical composition of claim 40, wherein the siRNA is targeted to human epidermal growth factor receptor 2 (HER2) mRNA.

48. The pharmaceutical composition of claim 47, wherein the sense strand of the siRNA is GGAGCUGGCGGCCUUGUGCCG (SEQ ID NO: 4).

49. The pharmaceutical composition of claim 47, wherein the sense strand of the siRNA is UCACAGGGGCCUCCCCAGGAG (SEQ ID NO: 5).

50. The pharmaceutical composition of claim 27, wherein the pharmaceutically acceptable carrier is solid.

51. The pharmaceutical composition of claim 27, wherein the pharmaceutically acceptable carrier is liquid.

52. The pharmaceutical composition of claim 51, wherein the pharmaceutically acceptable carrier comprises water.

53. The pharmaceutical composition of claim 51, wherein the pharmaceutically acceptable carrier is an isotonic salt solution.

54. The pharmaceutical composition of claim 51, wherein the pharmaceutically acceptable carrier is an isotonic sugar solution.

55. The pharmaceutical composition of claim 51, wherein the pharmaceutically acceptable carrier is an aqueous polyethylene glycol (PEG) solution.

56. The pharmaceutical composition of claim 51, wherein the pharmaceutically acceptable carrier is an organic solvent dissolved in isotonic aqueous solution.

57. The pharmaceutical composition of claim 51, wherein the pharmaceutically acceptable carrier is an aqueous buffer solution.

58. The pharmaceutical composition of claim 27, wherein the final concentrations of the pharmaceutical composition are 3 mg/L nonfunctionalized single-walled carbon nanotube and about 5 μM siRNA.

59. The pharmaceutical composition of claim 27, wherein said pharmaceutical composition provides delivery of an effective amount of said siRNA, and wherein said effective amount reduces the expression of a target nucleic acid when compared to siRNA not complexed to the nonfunctionalized single-walled carbon nanotube.

60. A method of reducing the expression of a targeted gene in cell culture, said method comprising: delivering an effective amount of a single-walled carbon nanotube composition to cells in said cell culure, wherein the composition comprises a nonfunctionalized single-walled carbon nanotube and a siRNA noncovalently complexed with the nonfunctionalized single-walled carbon nanotube, and wherein the siRNA solubilizes such nonfunctionalized single-walled carbon nanotube.

61. A method of effectively silencing a targeted gene in vivo, said method comprising: administering to a subject an effective amount of a single-walled carbon nanotube composition, wherein the composition comprises a nonfunctionalized single-walled carbon nanotube and a siRNA noncovalently complexed with the nonfunctionalized single-walled carbon nanotube, and wherein the siRNA solubilizes such nonfunctionalized single-walled carbon nanotube.

62. A method for preparing a single-walled carbon nanotube composition, said method comprising:
a) providing a dry nonfunctionalized single-walled carbon nanotube;
b) providing a siRNA solution;
c) adding the dry nonfunctionalized single-walled carbon nanotube to the siRNA solution; and
d) sonicating the nonfunctionalized single-walled carbon nanotube in the siRNA solution.

63. The method of claim 62, wherein the final concentration of the nonfunctionalized single-walled carbon nanotube in the siRNA solution is about 1 mg/L to about 5 mg/L, and wherein the final concentration of siRNA is about 3 μM to about 7 μM.

64. The method of claim 62, wherein the step of providing the siRNA solution comprises resuspending siRNA in solution.

65. The method of claim 64, wherein the solution comprises water.

66. The method of claim 64, wherein the solution is an isotonic salt solution.

67. The method of claim 64, wherein the solution is an isotonic sugar solution.

68. The method of claim 64, wherein the solution is an aqueous polyethylene glycol (PEG) solution.

69. The method of claim 64, wherein the solution is an organic solvent dissolved in isotonic aqueous solution.

70. The method of claim 64, wherein the solution is an aqueous buffer solution.

71. The method of claim 62, wherein the diameter of the nonfunctionalized single-walled carbon nanotube is about 1 nm to about 2 nm.

72. The method of claim 62, wherein the diameter of the nonfunctionalized single-walled carbon nanotube is about 1 nm.

73. The method of claim 62, wherein the length of the nonfunctionalized single-walled carbon nanotube is about 500 nm or less.

74. The method of claim 62, wherein the length of the nonfunctionalized single-walled carbon nanotube is about 400 nm or less.

75. The method of claim 62, wherein the length of the nonfunctionalized single-walled carbon nanotube is about 100 nm to about 300 nm.

76. The method of claim 62, wherein the length of the nonfunctionalized single-walled carbon nanotube is about 125 nm to about 275 nm.

77. The method of claim 62, wherein the length of the nonfunctionalized single-walled carbon nanotube is about 150 nm to about 250 nm.

78. The method of claim 62, wherein the length of the nonfunctionalized single-walled carbon nanotube is about 175 nm to about 225 nm.

79. The method of claim 62, wherein the siRNA comprises chemically-modified siRNA.

80. The method of claim 64, wherein the siRNA comprises stabilized siRNA.

81. The method of claim 62, wherein the siRNA comprises non-targeting siRNA.

82. The method of claim 64, wherein the siRNA comprises targeting siRNA.

83. The method of claim 82, wherein the siRNA is targeted to hypoxia-inducible factor 1 alpha (HIF-1α) mRNA.

84. The method of claim 82, wherein the siRNA is targeted to vascular endothelial growth factor (VEGF) mRNA.

85. The method of claim 84, wherein the sense strand of the siRNA is AUGUGAAUGCAGACCAAAGAA (SEQ ID NO: 1).

86. The method of claim 82, wherein the siRNA is targeted to endothelial growth factor receptor (EGFR) mRNA.

87. The method of claim 86, wherein the sense strand of the siRNA is GUCAGCCUGAACAUAACAU (SEQ ID NO: 2).

88. The method of claim 86, wherein the sense strand of the siRNA is GUGUAACGGAAUAGGUAUU (SEQ ID NO: 3).

89. The method of claim 82, wherein the siRNA is targeted to human epidermal growth factor receptor 2 (HER2) mRNA.

90. The method of claim 89, wherein the sense strand of the siRNA is GGAGCUGGCGGCCUUGUGCCG (SEQ ID NO: 4).

91. The method of claim 89, wherein the sense strand of the siRNA is UCACAGGGGCCUCCCCAGGAG (SEQ ID NO: 5).

92. A method for preparing a single-walled carbon nanotube composition comprising:
a) providing a dry nonfunctionalized single-walled carbon nanotube;
b) providing a siRNA solution;
c) adding the siRNA solution to the dry nonfunctionalized single-walled carbon nanotube; and
d) sonicating the nonfunctionalized single-walled carbon nanotube in the siRNA solution.

93. The method of claim 92, wherein the final concentration of the nonfunctionalized single-walled carbon nanotube in the siRNA solution is about 1 mg/L to about 5 mg/L nonfunctionalized single-walled carbon nanotube, and wherein the final concentration of siRNA is about 3 μM to about 7 μM.

94. The method of claim 92, wherein the step of providing the siRNA solution comprises resuspending siRNA in solution.

95. The method of claim 94, wherein the solution comprises water.

96. The method of claim 94, wherein the solution is an isotonic salt solution.

97. The method of claim 94, wherein the solution is an isotonic sugar solution.

98. The method of claim 94, wherein the solution is an aqueous polyethylene glycol (PEG) solution.

99. The method of claim 94, wherein the solution is an organic solvent dissolved in isotonic aqueous solution.

100. The method of claim 94, wherein the solution is an aqueous buffer solution.

101. The method of claim 92, wherein the diameter of the nonfunctionalized single-walled carbon nanotube is about 1 nm to about 2 nm.

102. The method of claim 92, wherein the diameter of the nonfunctionalized single-walled carbon nanotube is about 1 nm.

103. The method of claim 92, wherein the length of the nonfunctionalized single-walled carbon nanotube is about 500 nm or less.

104. The method of claim 92, wherein the length of the nonfunctionalized single-walled carbon nanotube is about 400 nm or less.

105. The method of claim 92, wherein the length of the nonfunctionalized single-walled carbon nanotube is about 100 nm to about 300 nm.

106. The method of claim 92, wherein the length of the nonfunctionalized single-walled carbon nanotube is about 125 nm to about 275 nm.

107. The method of claim 92, wherein the length of the nonfunctionalized single-walled carbon nanotube is about 150 nm to about 250 nm.

108. The method of claim 92, wherein the length of the nonfunctionalized single-walled carbon nanotube is about 175 nm to about 225 nm.

109. The method of claim 92, wherein the siRNA comprises chemically-modified siRNA.

110. The method of claim 92, wherein the siRNA comprises stabilized siRNA.

111. The method of claim 92, wherein the siRNA comprises non-targeting siRNA.

112. The method of claim 92, wherein the siRNA comprises targeting siRNA.

113. The method of claim 112, wherein the siRNA is targeted to hypoxia-inducible factor 1 alpha (HIF-1α) mRNA.

114. The method of claim 112, wherein the siRNA is targeted to vascular endothelial growth factor (VEGF) mRNA.

115. The method of claim 114, wherein the sense strand of the siRNA is AUGUGAAUGCAGACCAAAGAA (SEQ ID NO: 1).

116. The method of claim 112, wherein the siRNA is targeted to endothelial growth factor receptor (EGFR) mRNA.

117. The method of claim 116, wherein the sense strand of the siRNA is GUCAGCCUGAACAUAACAU (SEQ ID NO: 2).

118. The method of claim 116, wherein the sense strand of the siRNA is GUGUAACGGAAUAGGUAUU (SEQ ID NO: 3).

119. The method of claim 112, wherein the siRNA is targeted to human epidermal growth factor receptor 2 (HER2) mRNA.

120. The method of claim 119, wherein the sense strand of the siRNA is GGAGCUGGCGGCCUUGUGCCG (SEQ ID NO: 4).

121. The method of claim 119, wherein the sense strand of the siRNA is UCACAGGGGCCUCCCCAGGAG (SEQ ID NO: 5).


SEQUENCE LISTING (18 sequences)

SEQ ID NO: 1 - RNA, 21 nt, Homo sapiens
augugaaugcagaccaaagaa

SEQ ID NO: 2 - RNA, 19 nt, Homo sapiens
gucagccugaacauaacau

SEQ ID NO: 3 - RNA, 19 nt, Homo sapiens
guguaacggaauagguauu

SEQ ID NO: 4 - RNA, 21 nt, Homo sapiens
ggagcuggcggccuugugccg

SEQ ID NO: 5 - RNA, 21 nt, Homo sapiens
ucacaggggccuccccaggag

SEQ ID NO: 6 - RNA, 19 nt, Homo sapiens
ccugugucuaaaucugaac

SEQ ID NO: 7 - RNA, 20 nt, Homo sapiens
cuaccuucgugauucuguuu

SEQ ID NO: 8 - RNA, 19 nt, Homo sapiens
gcacaauagacagcgaaac

SEQ ID NO: 9 - RNA, 19 nt, Homo sapiens
cuacuuucuuaauggcuua

SEQ ID NO: 10 - RNA, 22 nt, Homo sapiens
caaccaaagucgaauauugauu

SEQ ID NO: 11 - RNA, 21 nt, Homo sapiens
caagaagaaugaauacaguuu

SEQ ID NO: 12 - RNA, 21 nt, Homo sapiens
gaagauguccauggaaauauu

SEQ ID NO: 13 - RNA, 21 nt, Homo sapiens
caacacgccucauccucuauu

SEQ ID NO: 14 - RNA, 19 nt, Homo sapiens
cgucuuuagauuccuauau

SEQ ID NO: 15 - RNA, 19 nt, Homo sapiens
guuguuccuacuucagaua

SEQ ID NO: 16 - RNA, 19 nt, Homo sapiens
gucgucuuuagauuccuau

SEQ ID NO: 17 - RNA, 19 nt, Homo sapiens
gaucuaccgaaagagucau

SEQ ID NO: 18 - RNA, 19 nt, Homo sapiens
uagcgacauuuguguaguu